SEQUENCE LISTING (12 sequences)

SEQ ID NO: 1 - protein, 5 aa, synthetic construct
DYYIH

SEQ ID NO: 2 - protein, 17 aa, synthetic construct
RVNPDHGDSYYNQKFKD

SEQ ID NO: 3 - protein, 6 aa, synthetic construct
NYLFDH

SEQ ID NO: 4 - protein, 11 aa, synthetic construct
KASQDVGTAVA

SEQ ID NO: 5 - protein, 7 aa, synthetic construct
WASIRHT

SEQ ID NO: 6 - protein, 7 aa, synthetic construct
HQFATYT

SEQ ID NO: 7 - protein, 115 aa, synthetic construct
EVQLVQSGAEVKKPGATVKISCKVSGYTFTDYYIHWVQQAPGKGLEWMGRVNPDHGDSYYNQKFKDKATITADKSTDTAYMELSSLRSEDTAVYFCARNYLFDHWGQGTLVTVSS

SEQ ID NO: 8 - protein, 105 aa, synthetic construct
DIQMTQSPSSVSASVGDRVTITCKASQDVGTAVAWYQQKPGKAPKLLIYWASIRHTGVPSRFSGSGSGTDFTLTISSLQPEDFATYYCHQFATYTFGGGTKVEIK

SEQ ID NO: 9 - protein, 444 aa, synthetic construct
EVQLVQSGAEVKKPGATVKISCKVSGYTFTDYYIHWVQQAPGKGLEWMGRVNPDHGDSYYNQKFKDKATITADKSTDTAYMELSSLRSEDTAVYFCARNYLFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 445 aa, synthetic construct
EVQLVQSGAEVKKPGATVKISCKVSGYTFTDYYIHWVQQAPGKGLEWMGRVNPDHGDSYYNQKFKDKATITADKSTDTAYMELSSLRSEDTAVYFCARNYLFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 212 aa, synthetic construct
DIQMTQSPSSVSASVGDRVTITCKASQDVGTAVAWYQQKPGKAPKLLIYWASIRHTGVPSRFSGSGSGTDFTLTISSLQPEDFATYYCHQFATYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 9 aa, synthetic construct
ARNYLFDHW

(19) United States
(12) Patent Application Publication
(10) Pub. No.: US 2026/0158154 A1
(43) Pub. Date: Jun. 11, 2026

(54) Methods of Treating Cancer Using Anti-HER2 Antibody-Drug Conjugates

(71) Applicant: Seagen Inc., Bothell, WA (US)

(72) Inventors: Nicholas Wei Wen Choong, Bothell, WA (US); Xuemei Li, Kirkland, WA (US)

(73) Assignee: Seagen Inc., Bothell, WA (US)

(21) Appl. No.: 19/537,666

(22) Filed: Feb. 12, 2026

Related U.S. Application Data
(63) Continuation of application No. PCT/US2024/042115, filed on Aug. 13, 2024.
(60) Provisional application No. 63/519,542, filed on Aug. 14, 2023.
(60) Provisional application No. 63/625,162, filed on Jan. 25, 2024.

(51) Int. Cl. A61K 47/68; C07K 16/32

(52) U.S. Cl. CPC A61K 47/68031; A61K 47/6855; C07K 16/32

(57) ABSTRACT

Provided herein are methods for treating or preventing progression of cancer in an individual, comprising administering to the individual an effective amount of an anti-human epidermal growth factor receptor 2 (HER2) antibody-drug conjugate that comprises an anti-HER2 antibody and a cytotoxic molecule.

### CROSS-REFERENCE TO RELATED APPLICATIONS

This application is a continuation application of International Application No. PCT/US2024/042115, filed on Aug. 13, 2024, which claims the priority benefit of U.S. Provisional Application No. 63/519,542, filed on Aug. 14, 2023, and U.S. Provisional Application No. 63/625,162, filed on Jan. 25, 2024, the entire contents of each of which are incorporated herein by reference for all purposes.

### REFERENCE TO AN ELECTRONIC SEQUENCE LISTING

This application contains a sequence listing, which has been submitted electronically in XML format. Said XML copy, created on Aug. 9, 2024, is named “01218-0044-00PCT.xml” and is 12,701 bytes in size. The information in the sequence listing is incorporated herein by reference in its entirety for all purposes.

### FIELD

The present disclosure relates to methods for treating or preventing progression of a cancer in an individual, as well as compositions, uses, and kits related thereto. In some embodiments, the methods comprise administering to the individual an effective amount of an anti-human epidermal growth factor receptor 2 (HER2) antibody-drug conjugate that comprises an anti-HER2 antibody and a cytotoxic molecule.

### BACKGROUND

ErbB2, also known as HER2/neu, is a member of a family of tyrosine kinases that regulate cell growth and survival (Lengyel, C. G. et al. (2021) Gastrointest Disord 3(1):1-22). Overexpression and/or amplification of HER2 is seen in many malignancies including breast, gastric, ovarian, pancreatic, colorectal, and endometrial cancer (Neve, R. M. et al. (2001) Ann Oncol 12(Suppl1):S9-S13; Menard, S. et al. (2003) Oncogene 22(42):6570-6578; Moasser, M. M. (2007) Oncogene 26(45):6469-6487; Iqbal, N. et al. (2014) Mol Biol Int 2014:852748). Tumors that overexpress HER2 are thought to be more aggressive and associated with poorer overall survival (OS) compared to HER2-negative cancers. Cancers characterized by overexpression of HER2 (e.g., HER2-positive cancers) are often correlated with poor prognosis and/or are resistant to many standard therapies. As such, a handful of therapies targeting HER2 have been approved for treatment of HER2-positive cancer, including antibodies such as trastuzumab and pertuzumab, antibody-drug conjugates (ADCs) such as trastuzumab maytansine (T-DM1), and small molecule tyrosine kinase inhibitors (TKIs) such as lapatinib, pazopanib, afatinib, and neratinib.

The introduction of HER2-targeted therapy using either antibody-based therapies or small molecule tyrosine kinase inhibitors (TKI) has led to significant and ongoing improvements in disease-free survival (DFS), progression-free survival (PFS), and OS in both the neoadjuvant/adjuvant and metastatic settings (Slamon, D. J. et al. (2001) N Engl J Med 344(11):783-792; Geyer, C. E. et al. (2006) N Engl J Med 355 (26): 2733-2743; Baselga 2012; Verma 2012). HER2 is an effective therapeutic target in multiple solid tumors with anti-HER2 biologic agents, antibody-drug conjugates (ADC), and small molecule drugs approved for patients with HER2 overexpressing/amplified (hereafter HER2+) breast and gastric cancers. Recently, interest has grown in HER2-targeting strategies for patients with HER2-low tumors, defined as those that are IHC 2+ and in situ hybridization (ISH) negative (−), or IHC 1+ following the approval of trastuzumab deruxtecan in HER2-low breast cancer. HER2 expression occurs commonly in many tumors. Table 1 outlines HER2 expression being present in up to 50% of patients for various disease types.

TABLE 1
Incidence of HER2 Expression in Solid Tumors

| Tumor Type | HER2 IHC 3+ | HER2 IHC 1+-3+^{a} |
|---|---|---|
| Salivary gland cancer | 20.0% | ~50% |
| Mesothelioma | 6.3% | ~20% |
| Cholangiocarcioma | 6.3% | ~50% |
| Uterine cancer | 3.0% | ~50% |
| NSCLC (squamous) | 2.0% | ~20% |
| Colorectal cancer | 1.8% | ~25%-50% |
| Ovarian cancer | 1.6% | ~25%-50% |
| HNSCC | 1.3% | ~20% |
| NSCLC (non-squamous) | 1.1% | ~50% |
| Gastroesophageal cancer | 1.0% | ~50% |
| Pancreatic cancer | 0.7% | ~25%-50% |
| Prostate cancer | 0.6% | ~25%-50% |
| Hepatocellular carcinoma | 0.4% | ~20% |

ERBB2 = erb-b2 receptor tyrosine kinase 2; HER2 = human epidermal growth factor receptor 2; HNSCC = head and neck squamous cell carcinoma; IHC = immunohistochemistry; NSCLC = non-small cell lung cancer.
^{a}Estimated from ERBB2 RNA expression using TEMPUS Labs real-world data.

With the development of ADC, HER2 expression can be utilized as target to direct the cytotoxic drug to the tumor cells. After binding of an HER2 directed ADC to HER2 on the cell surface, the ADC-receptor complex internalizes and traffics through the endo-lysosomal pathway. The subsequent release of cytotoxic payload eventually leads to cell apoptosis. New generations of ADCs with cleavable linkers may exhibit a bystander effect and have shown encouraging single agent activity in multiple HER2 expressing advanced tumors including breast cancer, gastric cancer, urothelial cancer. The data on HER2 directed ADCs observed to date suggest the potential for clinically meaningful efficacy in HER2 expressing tumors, and safety and efficacy data demonstrated a favorable benefit-risk profile which supports further development of HER2 directed ADCs in a broader range of tumor types with HER2 expression. Currently, there are no approved therapies, or even existing randomized/controlled phase 3 clinical data for HER2-directed ADCs, for HER2 expressing head and neck squamous cell carcinoma (HNSCC), NSCLC, ovarian cancer, and endometrial cancer.

In 2019, head and neck cancers (HNCs) have affected more than 3.8 million people worldwide (mouth 1.4 million, throat 1.2 million, and larynx 1.2 million), and caused over 507,600 deaths, with over 900,000 new cases arising per year (GBD 2019 Diseases and Injuries Collaborators (2019) Lancet. 2020; 396(10258):1204-22). Only 40% to 50% of patients will survive for 5 years (Leemans C. R., et al. (2011) Nat Rev Cancer 11(1):9-22). HNSCCs develop from the mucosal epithelium in the oral cavity, pharynx and larynx and are the most common malignancies that arise in the head and neck.

For patients with recurrent and/or metastatic HNSCC, systemic therapies are often used. Pembrolizumab in combination with platinum and fluoropyrimidine (FU) is the standard first-line treatment option for patients with metastatic or with unresectable, recurrent HNSCC. Following first-line therapy, cytotoxic chemotherapies such as methotrexate, fluorouracil (5-FU), or taxanes-either alone or in combination- and the biologic agent cetuximab are utilized. Patients whose disease progresses after platinum-containing chemotherapy have a poor prognosis. Despite these treatment options, metastatic or recurrent HNC remains an area of high unmet medical need as patients who progress after treatment (refractory or platinum-resistant disease) have the worst prognosis with median overall survival (OS) of 3 to 4 months and 1 year survival rate of <5% (León X, et al. (2005) Clin Oncol (R Coll Radiol) 17(6):418-24; Vermorken J. B. et al. (2007) J Clin Oncol 25(16):2171-7). Developing new effective therapy is urgently needed to improve clinical outcome of these patients.

Lung cancer is the leading cause of cancer deaths in the US. Non-small cell lung cancer (NSCLC) can be subclassified as squamous (approximately 20-30% of NSCLC cases) and non-squamous (approximately 80% of NSCLC cases) histological types. Squamous NSCLC is a distinct histological subtype of NSCLC that is challenging to treat as a result of specific patient and disease characteristics, which include older age, advanced disease at diagnosis, comorbid disease, and the central location of tumors (Socinski M. A., et al. (2018) J Thorac Oncol. 13(2):165-83). These characteristics have a bearing on treatment outcomes in advanced squamous NSCLC, resulting in a median survival rate of approximately 30% shorter than for patients with other NSCLC subtypes. Non-squamous NSCLC is a heterogenous disease with multiple treatment options dependent upon staging, presence of metastasis, and patient factors, including presence of comorbidities among other considerations. As such, treatment options include surgical resection, chemotherapy, radiation, immunotherapy, and targeted therapy. Treatment recommendations depend heavily on the existence or absence of driver mutations and/or PD-L1 expression (Hanna, N. H., et al. (2020) J Clin Oncol. 38(14):1608-32).

For patients who present with advanced disease without targetable driver mutations (e.g., EGFR mutation or ALK translocation), including those who present with metastases (stage IV) or recur following initial definitive treatment, cytotoxic platinum-based chemotherapy combined with a PD-(L)1 targeted agent is currently standard of care first line therapy regardless of PD-L1 expression levels. In patients with high PD-L1 expression, anti-PD-(L)1-single agent treatment may be considered (Hellmann, M. D. et al. (2019) N Engl J Med. 381 (21): 2020-31; Zhang, C. et al, (2019) J Hematol Oncol. 12(1):45). With the improved clinical benefit seen with chemotherapy and PD-(L)1-targeted (+/−CTLA4 inhibitor) agents in frontline treatment of NSCLC without targetable mutations, the benefit from later lines of therapy is considered to be limited, leaving patients without effective treatment options once they progress on a PD-(L)1-targeted agent or develop resistance to platinum-based chemotherapy. A clinical trial to evaluate the efficacy and safety of disitamab vedotin for subjects with advanced non-small cell lung cancer with HER2 overexpression (IHC≥1+) or HER2 mutation is on-going with subjects treated intravenously with a dose of 2.0 mg/kg (measured via a bovine serum albumin (BSA)-based extinction coefficient method) every 2 weeks (www.clinicaltrials.gov/ct2/show/NCT04311034). New treatment options for patients who have exhausted the available PD-(L)1-targeted agents and cytotoxic platinum-based chemotherapy are urgently needed to improve clinical outcome of these patients.

Ovarian carcinoma is the second most common gynecologic malignancy (second to uterine carcinoma) and the most common cause of gynecologic cancer death in the US and other resource-abundant countries. The majority of ovarian malignancies (95%) are derived from epithelial cells (subtypes include high-grade serous, low-grade serous, endometrioid, clear cell, and mucinous); the remainder arise from other ovarian cell types (germ cell tumors, sex cord-stromal tumors; Chen L M, Berek J S. Epithelial carcinoma of the ovary, fallopian tube, and peritoneum: clinical features and diagnosis. UpToDate. Accessed Feb. 1, 2023). High-grade serous epithelial ovarian carcinoma, fallopian tube, and peritoneal carcinomas are considered a single clinical entity due to their shared clinical behavior and treatment. There is also accumulating evidence of a common pathogenesis for these carcinomas.

After initial surgical diagnosis, staging and cytoreduction, the standard primary systemic chemotherapy for women with advanced epithelial ovarian and peritoneal primary cancer consists of chemotherapy with a platinum and taxane combination, usually carboplatin and paclitaxel (du Bois, A. et al. (2003) J Natl Cancer Inst. 95(17):1320-9; Ozols, R. F. et al (2003) J Clin Oncol. 2003; 21(17):3194-200). Although most patients may initially experience response to platinum-based chemotherapy, the majority will relapse. Patients relapsing within 6 months of platinum therapy (platinum resistant) have a poorer response to subsequent treatments than those relapsing later, and as a result have a decreased OS. Over the past two decades, there have been only modest improvements in 5-year OS for women with advanced-stage ovarian tumors. Improvements are needed in therapeutic strategies in treating advanced stage ovarian cancer.

Cancer of the endometrium of the uterus is the most common gynecologic malignancy in resource-abundant countries and the second most common in resource-limited countries (cervical cancer is more common). Endometrioid carcinoma (EMC) is the most common histologic type of endometrial carcinoma and of uterine malignancy overall. Endometrioid tumors tend to have a favorable prognosis and typically present at an early stage with abnormal uterine bleeding. Other histologic types of endometrial carcinoma (eg, serous, clear cell) as well as other types of uterine cancer are associated with a poor prognosis.

For patients presenting with advanced disease, treatment options are limited with no consensus on a standard regimen. Chemotherapy has been the standard of care in the first-line treatment of EMC, with platinum compounds, anthracyclines, and taxanes being the most commonly used, alone and in combination (Colombo, P. E. et al. (2013) Ann Oncol. 2013; 24(Suppl 6):vi33-8). Cytotoxic therapy is a treatment option in second-line treatment; however, response rates are low, PFS and OS are short. There are limited subsequent treatment options in relapsed/refractory patients, including: single agent doxorubicin, paclitaxel, pegylated liposomal doxorubicin, and bevacizumab. The median OS in clinical trials after first- or second-line agents is generally 12 months or less. There remains an unmet clinical need for second-line or later line therapies in this patient population.

Patients with locally-advanced, unresectable or metastatic (LA/m) head and neck squamous cell carcinoma (HNSCC), non-small cell lung cancer (NSCLC), ovarian cancer, and endometrial cancer have limited treatment options after front line therapy. Second line and subsequent treatments often result in low response rates and modest duration of disease control. In addition, human epidermal growth factor receptor 2 (HER2) is expressed in a significant proportion of these tumors but therapies targeting such HER2-expressing tumors have not been developed. As such, there remains a need for novel therapies in the late-line setting that can provide clinical benefit, particularly for treatment of LA/m head and neck squamous cell carcinoma (HNSCC), non-small cell lung cancer (NSCLC), ovarian cancer, and endometrial cancer.

All references cited herein, including patent applications, patent publications, and UniProtKB/Swiss-Prot Accession numbers are herein incorporated by reference in their entirety, as if each individual reference were specifically and individually indicated to be incorporated by reference.

### SUMMARY

In some aspects, provided herein are methods for treating or preventing progression of cancer in an individual, comprising administering to the individual an effective amount of an anti-human epidermal growth factor receptor 2 (HER2) antibody-drug conjugate that comprises an anti-HER2 antibody and a cytotoxic molecule; wherein the anti-HER2 antibody comprises a heavy chain comprising a heavy chain variable (VH) domain and a light chain comprising a light chain variable (VL) domain; wherein the VH domain comprises a CDR-H1 comprising the amino acid sequence DYYIH (SEQ ID NO:1), a CDR-H2 comprising the amino acid sequence RVNPDHGDSYYNQKFKD (SEQ ID NO:2), and a CDR-H3 comprising the amino acid sequence NYLFDH (SEQ ID NO:3); and wherein the VL domain comprises a CDR-L1 comprising the amino acid sequence KASQDVGTAVA (SEQ ID NO: 4), a CDR-L2 comprising the amino acid sequence WASIRHT (SEQ ID NO:5), and a CDR-L3 comprising the amino acid sequence HQFATYT (SEQ ID NO:6); and wherein the cancer is selected from the group consisting of: head and neck squamous cell carcinoma (HNSCC), ovarian cancer, and endometrial cancer.

In some aspects, provided herein are methods for treating or preventing progression of cancer in an individual, comprising administering to the individual an effective amount of an anti-human epidermal growth factor receptor 2 (HER2) antibody-drug conjugate that comprises an anti-HER2 antibody and a cytotoxic molecule; wherein the anti-HER2 antibody comprises a heavy chain comprising a heavy chain variable (VH) domain and a light chain comprising a light chain variable (VL) domain; wherein the VH domain comprises a CDR-H1 comprising the amino acid sequence DYYIH (SEQ ID NO:1), a CDR-H2 comprising the amino acid sequence RVNPDHGDSYYNQKFKD (SEQ ID NO:2), and a CDR-H3 comprising the amino acid sequence ARNYLFDHW (SEQ ID NO:12); and wherein the VL domain comprises a CDR-L1 comprising the amino acid sequence KASQDVGTAVA (SEQ ID NO: 4), a CDR-L2 comprising the amino acid sequence WASIRHT (SEQ ID NO:5), and a CDR-L3 comprising the amino acid sequence HQFATYT (SEQ ID NO:6); and wherein the cancer is selected from the group consisting of: head and neck squamous cell carcinoma (HNSCC), ovarian cancer, and endometrial cancer.

In some aspects, provided herein are methods for treating or preventing progression of a cancer in an individual, comprising administering to the individual an effective amount of an anti-human epidermal growth factor receptor 2 (HER2) antibody-drug conjugate that comprises an anti-HER2 antibody and a cytotoxic molecule; wherein the anti-HER2 antibody comprises a heavy chain comprising a heavy chain variable (VH) domain and a light chain comprising a light chain variable (VL) domain; wherein the VH domain comprises a CDR-H1 comprising the amino acid sequence DYYIH (SEQ ID NO:1), a CDR-H2 comprising the amino acid sequence RVNPDHGDSYYNQKFKD (SEQ ID NO:2), and a CDR-H3 comprising the amino acid sequence NYLFDH (SEQ ID NO:3); and wherein the VL domain comprises a CDR-L1 comprising the amino acid sequence KASQDVGTAVA (SEQ ID NO: 4), a CDR-L2 comprising the amino acid sequence WASIRHT (SEQ ID NO:5), and a CDR-L3 comprising the amino acid sequence HQFATYT (SEQ ID NO:6); and wherein the cancer is non-small cell lung cancer (NSCLC), and wherein, prior to administration of the antibody-drug conjugate, the individual (a) progressed during or after a platinum-based therapy or, within 6 months of platinum-based adjuvant, neoadjuvant, or concomitant chemoradiotherapy for early or locally-advanced stage disease; or (b) received prior anti-PD(L)1 therapy. In some embodiments, prior to administration of the antibody-drug conjugate, the individual (a) progressed during or after a platinum-based therapy or, within 6 months of platinum-based adjuvant, neoadjuvant, or concomitant chemoradiotherapy for early or locally-advanced stage disease; and (b) received prior anti-PD(L)1 therapy.

In some aspects, provided herein are methods for treating or preventing progression of a cancer in an individual, comprising administering to the individual an effective amount of an anti-human epidermal growth factor receptor 2 (HER2) antibody-drug conjugate that comprises an anti-HER2 antibody and a cytotoxic molecule; wherein the anti-HER2 antibody comprises a heavy chain comprising a heavy chain variable (VH) domain and a light chain comprising a light chain variable (VL) domain; wherein the VH domain comprises a CDR-H1 comprising the amino acid sequence DYYIH (SEQ ID NO:1), a CDR-H2 comprising the amino acid sequence RVNPDHGDSYYNQKFKD (SEQ ID NO:2), and a CDR-H3 comprising the amino acid sequence ARNYLFDHW (SEQ ID NO:12); and wherein the VL domain comprises a CDR-L1 comprising the amino acid sequence KASQDVGTAVA (SEQ ID NO: 4), a CDR-L2 comprising the amino acid sequence WASIRHT (SEQ ID NO:5), and a CDR-L3 comprising the amino acid sequence HQFATYT (SEQ ID NO:6); and wherein the cancer is non-small cell lung cancer (NSCLC), and wherein, prior to administration of the antibody-drug conjugate, the individual (a) progressed during or after a platinum-based therapy or, within 6 months of platinum-based adjuvant, neoadjuvant, or concomitant chemoradiotherapy for early or locally-advanced stage disease; or (b) received prior anti-PD(L)1 therapy. In some embodiments, prior to administration of the antibody-drug conjugate, the individual (a) progressed during or after a platinum-based therapy or, within 6 months of platinum-based adjuvant, neoadjuvant, or concomitant chemoradiotherapy for early or locally-advanced stage disease; and (b) received prior anti-PD(L)1 therapy.

In some embodiments according to any of the embodiments described herein, the VH domain comprises the amino acid sequence of SEQ ID NO:7. In some embodiments according to any of the embodiments described herein, the VL domain comprises the amino acid sequence of SEQ ID NO:8. In some embodiments according to any of the embodiments described herein, the heavy chain comprises the amino acid sequence of SEQ ID NO:9 or SEQ ID NO:10. In some embodiments according to any of the embodiments described herein, the light chain comprises the amino acid sequence of SEQ ID NO:11.

In some embodiments according to any of the embodiments described herein, the cytotoxic molecule comprises a tubulin inhibitor or DNA damaging agent. In some embodiments, the tubulin inhibitor comprises a dolastatin or derivative thereof, auristatin or derivative thereof, or maytansinoid or derivative thereof. In some embodiments, the tubulin inhibitor comprises monomethyl auristatin E (MMAE), monomethyl auristatin F (MMAF), or auristatin F (AF). In some embodiments, the tubulin inhibitor comprises emtansine (DM1), maytansine (DM3), or ravtansine (DM4). In some embodiments, the DNA damaging agent comprises a calicheamicin, duocarmycin, pyrrolobenzodiazepine (PBD), or SN-38. In some embodiments according to any of the embodiments described herein, the cytotoxic molecule comprises an amanitin, anthracycline, baccatin, camptothecin, cemadotin, colchicine, colcemid, combretastatin, cryptophycin, discodermolide, docetaxel, doxorubicin, echinomycin, eleutherobin, epothilone, estramustine, lexitropsins, maytansine, methotrexate, netropsin, puromycin, rhizoxins, taxane, tubulysin, or vinca alkaloid. In some embodiments according to any of the embodiments described herein, the antibody-drug conjugate is represented by formula Ab-(L-U)n, wherein Ab is the anti-HER2 antibody, L is a linker between the cytotoxic molecule and the anti-HER2 antibody, U is the conjugated cytotoxic molecule, and n is an integer from 1 to 8, representing the number of cytotoxic molecules bound to the antibody. In some embodiments, the linker is attached to the anti-HER2 antibody via a thiol or amino moiety. In some embodiments, the linker is selected from the group consisting of maleimidocaproyl valine citrulline p-amino-benzyloxy (mc-vc-pAB), maleimidocaproyl (mc), triglycol peptide linker, 3-maleimido-propionic acid, Mal-di-EG-OPFP (perfluorophenyl 3-(2-(2-(3-(2,5-dioxo-2,5-dihydro-1H-pyrrol-1-yl) propanamido) ethoxy) ethoxy) propanoate), Mal-di-EG-OSu (2,5-dioxopyrrolidin-1-yl 3-(2-(2-(2,5-dioxo-2,5-dihydro-1H-pyrrol-1-yl) ethoxy) ethoxy) propanoate), Mal-Tri-EG-OSu (2,5-dioxopyrrolidin-1-yl 3-(2-(2-(2-(2,5-dioxo-2,5-dihydro-1H-pyrrol-1-yl) ethoxy) ethoxy) ethoxy) ethoxypropanoate), Mal-Tetra-EG-OSu (2,5-dioxopyrrolidin-1-yl 1-(2,5-dioxo-2,5-dihydro-1H-pyrrol-1-yl)-3-oxo-7,10,13,16-tetraoxa-4-azanonadecan-19-oate), Br-di-EG-OSu (2,5-dioxopyrrolidin-1-yl 3 (2-(2-(2-bromoacetamido) ethoxy) ethoxy) propanoate), Py-ds-prp-OSu (2-5-dioxopyrrolidin-1-yl 3-(pyridine-2-yldisulfanyl) propanoate), Py-ds-Prp-OPEP (perfluorophenyl 3-(pyridine-2-yldisulfanyl) propanoate), Py-ds-dmBut-OSu (2,5-dioxopyrrolidin-1-yl 4-methyl-4-(pyridine-2-yldisulfanyl) pentanoate, Py-ds-dmBut-OPF (perfluorophenyl 4-methyl-4-(pyridine-2-yldisulfanyl) pentanoate), SMCC (N-succinimidyl 4-(maleimidomethyl) cyclohexanecarboxylate), MBS (3-maleimidobenzoic acid N-hydroxysuccinimide ester), SATA (S—(N-succinimidyl)thioacetate), SPDP ((N-succinimidyl 3-(2-pyridyldithio) propionate), and SMPT ((N-succinimidyloxy carbonyl)-1-methyl-1-(2-pyridyldithio) toluene). In some embodiments, the antibody-drug conjugate is disitamab vedotin.

In some embodiments according to any of the embodiments described herein, the cancer has been previously treated. In some embodiments according to any of the embodiments described herein, the cancer has been previously treated with a HER2 directed antibody-drug conjugate. In some embodiments according to any of the embodiments described herein, the cancer is a locally-advanced, unresectable or metastatic (LA/m) cancer. In some embodiments according to any of the embodiments described herein, prior to administration of the antibody-drug conjugate, the individual (a) progressed during or after ≥1 prior line of systemic therapy for LA/m disease and (b) progressed during or after, or is intolerant of, the most recent line of systemic therapy. In some embodiments, the cancer is HNSCC with a primary tumor site arising from the oral cavity, oropharynx, hypopharynx, or larynx. In some embodiments, the cancer is NSCLC with a mutation in EGFR, ALK, or ROS, and the individual has received prior targeted therapy. In some embodiments, the cancer is ovarian cancer comprising epithelial cancers of ovarian, fallopian tube, or peritoneal origin. In some embodiments, the cancer is ovarian cancer with a BRCA mutation, and the individual has received prior treatment with a PARP inhibitor. In some embodiments, the cancer is adenocarcinoma of the endometrium. In some embodiments according to any of the embodiments described herein, cells of the cancer express HER2. In some embodiments according to any of the embodiments described herein, the cancer is a HER2-positive cancer. In some embodiments, a sample obtained from the individual comprises cancer cells that express HER2 on their cell surface at a level of IHC≥1+, as measured by IHC assay. In some embodiments according to any of the embodiments described herein, the cancer is a HER2-zero cancer. In some embodiments, a sample obtained from the individual comprises cancer cells that express HER2 on their cell surface at a level of IHC-0, as measured by IHC assay.

In some embodiments according to any of the embodiments described herein, the antibody-drug conjugate is administered to the individual at a dose of 1.5 mg/kg. In some embodiments, the antibody-drug conjugate is administered to the individual at a maximum dose of 150 mg. In some embodiments, the dose is measured using the disitamab vedotin-based Extinction Coefficient (EC) method. In some embodiments according to any of the embodiments described herein, the antibody-drug conjugate is administered intravenously to the individual. In some embodiments according to any of the embodiments described herein, the antibody-drug conjugate is administered to the individual every 2 weeks or every 14 days. In some embodiments, the antibody-drug conjugate is disitamab vedotin, and the method comprises intravenously administering disitamab vedotin to the individual at 1.5 mg/kg with a maximum dose of 150 mg every two weeks or every 14 days.

In some embodiments according to any of the embodiments described herein, administration of the antibody-drug conjugate results in a complete response (CR) or partial response (PR) in the individual. In some embodiments, administration of the antibody-drug conjugate results in a complete response (CR) or partial response (PR) in the individual within about 3 years. In some embodiments according to any of the embodiments described herein, the individual has one or more known HER2 mutations. In some embodiments according to any of the embodiments described herein, the individual is a human.

In some aspects, provided herein are uses of an anti-HER2 antibody-drug conjugate for manufacture of a medicament for treating or preventing progression of a previously treated cancer in an individual, wherein the use comprises administering an effective amount of the anti-HER2 antibody-drug conjugate according to the methods of any of the above-described methods.

In some aspects, provided herein are pharmaceutical compositions for use in treating a previously treated cancer in an individual, wherein the use comprises administering an effective amount of the anti-HER2 antibody-drug conjugate according to any of the any described methods.

### BRIEF DESCRIPTION OF THE DRAWINGS

FIG. 1 shows the schema for a phase 2 basket study of disitamab vedotin in adult subjects with previously treated, locally-advanced unresectable or metastatic solid tumors (e.g., head and neck squamous cell carcinoma (HNSCC), non-small cell lung cancer (NSCLC), ovarian cancer, endometrial cancer) that express HER2. HER2-human epidermal growth factor receptor 2; NSCLC=non-small cell lung cancer; IHC=immunohistochemistry.

### DETAILED DESCRIPTION

### I. Definitions

Before describing the invention in detail, it is to be understood that this invention is not limited to particular compositions or biological systems, which can, of course, vary. It is also to be understood that the terminology used herein is for the purpose of describing particular embodiments only, and is not intended to be limiting.

As used in this specification and the appended claims, the singular forms “a”, “an” and “the” include plural referents unless the content clearly dictates otherwise. Thus, for example, reference to “a molecule” optionally includes a combination of two or more such molecules, and the like.

The term “about” as used herein refers to the usual error range for the respective value readily known to the skilled person in this technical field. Reference to “about” a value or parameter herein includes (and describes) embodiments that are directed to that value or parameter per se.

It is understood that aspects and embodiments of the invention described herein include “comprising,” “consisting,” and “consisting essentially of” aspects and embodiments.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2^{nd} ed., 2002, CRC Press; The Dictionary of Cell and Molecular Biology, 3^{rd} ed., 1999, Academic Press; and the Oxford Dictionary Of Biochemistry And Molecular Biology, Revised, 2000, Oxford University Press, provide one of skill with a general dictionary of many of the terms used in this disclosure. For purposes of the present disclosure, the following terms are defined.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The terms “about” and “approximately” as used herein shall generally mean an acceptable degree of error for the quantity measured given the nature or precision of the measurements. Typical, exemplary degrees of error are within 20 percent (%), preferably within 10%, and more preferably within 5% of a given value or range of values. Any reference to “about X” specifically indicates at least the values X, 0.95×, 0.96×, 0.97×, 0.98×, 0.99×, 1.01×, 1.02×, 1.03×, 1.04×, and 1.05×. Thus, “about X” is intended to teach and provide written description support for a claim limitation of, e.g., “0.98×.” The terms “about” and “approximately,” particularly in reference to a given quantity, encompass and describe the given quantity itself.

Alternatively, in biological systems, the terms “about” and “approximately” may mean values that are within an order of magnitude, preferably within 5-fold, and more preferably within 2-fold of a given value. Numerical quantities given herein are approximate unless stated otherwise, meaning that the term “about” or “approximately” can be inferred when not expressly stated.

When “about” is applied to the beginning of a numerical range, it applies to both ends of the range. Thus, “from about 5 to 20%” is equivalent to “from about 5% to about 20%” When “about” is applied to the first value of a set of values, it applies to all values in that set. Thus, “about 7, 9, or 11 mg/kg” is equivalent to “about 7, about 9, or about 11 mg/kg.”

“Administering” or “administration” refer to the physical introduction of a therapeutic agent to a subject, using any of the various methods and delivery systems known to those skilled in the art. Exemplary routes of administration include oral, intravenous, intramuscular, subcutaneous, intraperitoneal, spinal or other parenteral routes of administration, for example by injection or infusion (e.g., intravenous infusion). The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. A therapeutic agent can be administered via a non-parenteral route, or orally. Other non-parenteral routes include a topical, epidermal or mucosal route of administration, for example, intranasally, vaginally, rectally, sublingually or topically. Administration can also be performed, for example, once, a plurality of times, and/or over one or more extended periods.

A “cancer” refers to a broad group of various diseases characterized by the uncontrolled growth of abnormal cells in the body. A “cancer” or “cancer tissue” can include a tumor.

The term “metastasis” is an art known term that refers to the spread of cancer cells from the place where they first formed (the primary site) to one or more other sites in a subject (one or more secondary sites). In metastasis, cancer cells break away from the original (primary) tumor, travel through the blood or lymph system, and form a new tumor (a metastatic tumor) in other organs or tissues of the body. The new, metastatic tumor includes the same or similar cancer cells as the primary tumor. At the secondary site, the tumor cell may proliferate and begin the growth or colonization of a secondary tumor at this distant site.

The term “HER2” (also known as also known as HER2/neu, ERBB2, CD340, receptor tyrosine-protein kinase erbB-2, proto-oncogene Neu, and human epidermal growth factor receptor 2) refers to a member of the human epidermal growth factor receptor (HER/EGFR/ERBB) family of receptor tyrosine kinases. Amplification or overexpression of HER2 plays a significant role in the development and progression of certain aggressive types of cancer, including colorectal cancer, gastric cancer, lung cancer (e.g., non-small cell lung cancer (NSCLC)), biliary cancers (e.g., cholangiocarcinoma, gallbladder cancer), bladder cancer, esophageal cancer, melanoma, ovarian cancer, liver cancer, prostate cancer, pancreatic cancer, small intestine cancer, head and neck cancer, uterine cancer, cervical cancer, and breast cancer. Non-limiting examples of HER2 nucleotide sequences are set forth in GenBank reference numbers NP_001005862, NP_001289936, NP_001289937, NP 001289938, and NP_004448. Non-limiting examples of HER2 peptide sequences are set forth in GenBank reference numbers NP OO 1005862, NP_001276865, NP_001276866, NP_001276867, and NP_004439.

The term “anti-HER2 antibody-drug conjugate” refers to an anti-HER2 antibody conjugated to a therapeutic agent (i.e., a drug or cytotoxic molecule) optionally via a linker.

An “anti-HER2 antibody”, as used herein, refers to an antibody that binds to the HER2 protein. Anti-HER2 antibodies used for the treatment of cancer are typically monoclonal, although polyclonal antibodies are not excluded by the term. Anti-HER2 antibodies inhibit HER2 activation or downstream signaling by various mechanisms. As non-limiting examples, anti-HER2 antibodies can prevent ligand binding, receptor activation or receptor signal propagation, result in reduced HER2 expression or localization to the cell surface, inhibit HER2 cleavage, or induce antibody-mediated cytotoxicity. Non-limiting examples of anti-HER2 antibodies that are suitable for use in the methods and compositions of the present invention include hertuzumab, trastuzumab, pertuzumab, margetuximab, and combinations thereof.

“Treatment” or “therapy” of a subject refers to any type of intervention or process performed on, or the administration of an active agent to, the subject with the objective of reversing, alleviating, ameliorating, inhibiting, slowing down, or preventing the onset, progression, development, severity, or recurrence of a symptom, complication, condition, or biochemical indicia associated with a disease. In some embodiments, the disease is cancer. As used herein, the terms “treatment” and “treating” when referring, e.g., to the treatment of a cancer, are not intended to be absolute terms. For example, “treatment of cancer” and “treating cancer”, as used in a clinical setting, is intended to include obtaining beneficial or desired clinical results and can include an improvement in the condition of a subject having cancer. Beneficial or desired clinical results include, but are not limited to, one or more of the following: reducing the proliferation of (or destroying) neoplastic or cancerous cells, inhibiting metastasis of neoplastic cells, a decrease in metastasis in a subject, shrinking or decreasing the size of a tumor, change in the growth rate of one or more tumor(s) in a subject, an increase in the period of remission for a subject (e.g., as compared to the one or more metric(s) in a subject having a similar cancer receiving no treatment or a different treatment, or as compared to the one or more metric(s) in the same subject prior to treatment), decreasing symptoms resulting from a disease, increasing the quality of life of those suffering from a disease (e.g., assessed using FACT-G or EORTC-QLQC30), decreasing the dose of other medications required to treat a disease, delaying the progression of a disease, and/or prolonging survival of subjects having a disease.

The term “prophylactic” or “prophylactically” refers to any type of intervention or process performed on, or the administration of an active agent to, the subject with the objective of protecting or preventing a disease or condition from developing or at least not developing fully (e.g., to reduce the symptoms or severity of the disease or condition) such as in the development of a side effect (e.g., diarrhea).

A “subject” includes any human or non-human animal. The term “non-human animal” includes, but is not limited to, vertebrates such as non-human primates, sheep, dogs, and rodents such as mice, rats, and guinea pigs. In some embodiments, the subject is a human. The terms “subject” and “patient” and “individual” are used interchangeably herein.

An “effective amount” or “therapeutically effective amount” or “therapeutically effective dosage” of a drug or therapeutic agent is any amount of the drug that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

By way of example, an “anti-cancer agent” promotes cancer regression in a subject. In some embodiments, a therapeutically effective amount of the drug promotes cancer regression to the point of eliminating the cancer. “Promoting cancer regression” means that administering an effective amount of the drug, alone or in combination with an anti-cancer agent, results in a reduction in tumor growth or size, necrosis of the tumor, a decrease in severity of at least one disease symptom, an increase in frequency and duration of disease symptom-free periods, or a prevention of impairment or disability due to the disease affliction. In addition, the terms “effective” and “effectiveness” with regard to a treatment includes both pharmacological effectiveness and physiological safety. Pharmacological effectiveness refers to the ability of the drug to promote cancer regression in the patient. Physiological safety refers to the level of toxicity or other adverse physiological effects at the cellular, organ and/or organism level (adverse effects) resulting from administration of the drug.

The phrase “pharmaceutically acceptable” indicates that the substance or composition must be compatible chemically and/or toxicologically, with the other ingredients comprising a formulation, and/or the mammal being treated therewith.

As used herein, the term “pharmaceutically acceptable carrier” refers to a substance that aids the administration of an active agent to a cell, an organism, or a subject. “Pharmaceutically acceptable carrier” refers to a carrier or excipient that can be included in the compositions of the disclosure and that causes no significant adverse toxicological effect on the subject. Non-limiting examples of pharmaceutically acceptable carriers include water, NaCl, normal saline solutions, lactated Ringer's, normal sucrose, normal glucose, binders, fillers, disintegrants, lubricants, coatings, sweeteners, flavors and colors, liposomes, dispersion media, microcapsules, cationic lipid carriers, isotonic and absorption delaying agents, and the like. The carrier may also be substances for providing the formulation with stability, sterility and isotonicity (e.g., antimicrobial preservatives, antioxidants, chelating agents and buffers), for preventing the action of microorganisms (e.g. antimicrobial and antifungal agents, such as parabens, chlorobutanol, phenol, sorbic acid and the like) or for providing the formulation with an edible flavor etc. In some instances, the carrier is an agent that facilitates the delivery of a small molecule drug or antibody to a target cell or tissue. One of skill in the art will recognize that other pharmaceutical carriers are useful in the present disclosure.

The phrase “pharmaceutically acceptable salt” as used herein, refers to pharmaceutically acceptable organic or inorganic salts of a compound of the disclosure. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate”, ethanesulfonate, benzenesulfonate, p-toluenesulfonate, pamoate (i.e., 1,1′-methylene-bis(2-hydroxy-3-naphthoate)) salts, alkali metal (e.g., sodium and potassium) salts, alkaline earth metal (e.g., magnesium) salts, and ammonium salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

### II. Methods of Treating Cancer

Certain aspects of the present disclosure relate to methods for treating or preventing progression of cancer (e.g., head and neck squamous cell carcinoma (HNSCC), ovarian cancer, endometrial cancer, non-small cell lung cancer (NSCLC)) in an individual, comprising administering to the individual an effective amount of an anti-human epidermal growth factor receptor 2 (HER2) antibody-drug conjugate. In some embodiments, the cancer is head and neck squamous cell carcinoma (HNSCC), ovarian cancer, or endometrial cancer. In embodiments where the cancer is NSCLC, prior to administration of the antibody-drug conjugate, the individual has (a) progressed during or after a platinum-based therapy or, within 6 months of platinum-based adjuvant, neoadjuvant, or concomitant chemoradiotherapy for early or locally-advanced stage disease; or (b) received prior anti-PD(L)1 therapy. In embodiments where the cancer is NSCLC, prior to administration of the HER2-directed antibody-drug conjugate, the individual has (a) progressed during or after a platinum-based therapy or, within 6 months of platinum-based adjuvant, neoadjuvant, or concomitant chemoradiotherapy for early or locally-advanced stage disease; or (b) received prior anti-PD(L)1 therapy. In some embodiments, the cancer has previously been treated.

### Antibody-Drug Conjugates

In some embodiments, the anti-HER2 antibody comprises a heavy chain comprising a heavy chain variable (VH) domain and a light chain comprising a light chain variable (VL) domain. In some embodiments, the VH domain comprises a CDR-H1 comprising the amino acid sequence DYYIH (SEQ ID NO:1), a CDR-H2 comprising the amino acid sequence RVNPDHGDSYYNQKFKD (SEQ ID NO:2), and a CDR-H3 comprising the amino acid sequence NYLFDH (SEQ ID NO:3) or ARNYLFDHW (SEQ ID NO: 12); and/or the VL domain comprises a CDR-L1 comprising the amino acid sequence KASQDVGTAVA (SEQ ID NO:4), a CDR-L2 comprising the amino acid sequence WASIRHT (SEQ ID NO:5), and a CDR-L3 comprising the amino acid sequence HQFATYT (SEQ ID NO:6). In some embodiments, the VH domain comprises a CDR-H1 comprising the amino acid sequence DYYIH (SEQ ID NO:1), a CDR-H2 comprising the amino acid sequence RVNPDHGDSYYNQKFKD (SEQ ID NO:2), and a CDR-H3 comprising the amino acid sequence NYLFDH (SEQ ID NO:3); and the VL domain comprises a CDR-L1 comprising the amino acid sequence KASQDVGTAVA (SEQ ID NO:4), a CDR-L2 comprising the amino acid sequence WASIRHT (SEQ ID NO:5), and a CDR-L3 comprising the amino acid sequence HQFATYT (SEQ ID NO:6). In some embodiments, the VH domain comprises a CDR-H1 comprising the amino acid sequence DYYIH (SEQ ID NO:1), a CDR-H2 comprising the amino acid sequence RVNPDHGDSYYNQKFKD (SEQ ID NO:2), and a CDR-H3 comprising the amino acid sequence ARNYLFDHW (SEQ ID NO:12); and the VL domain comprises a CDR-L1 comprising the amino acid sequence KASQDVGTAVA (SEQ ID NO: 4), a CDR-L2 comprising the amino acid sequence WASIRHT (SEQ ID NO:5), and a CDR-L3 comprising the amino acid sequence HQFATYT (SEQ ID NO:6).

In some embodiments, the anti-HER2 antibody comprises a VH domain that comprises the amino acid sequence of SEQ ID NO:7 and/or a VL domain that comprises the amino acid sequence of SEQ ID NO:8. In some embodiments, the anti-HER2 antibody comprises a VH domain that comprises the amino acid sequence of SEQ ID NO:7 and a VL domain that comprises the amino acid sequence of SEQ ID NO:8.

In some embodiments, the anti-HER2 antibody comprises a heavy chain that comprises the amino acid sequence of SEQ ID NO:9 or SEQ ID NO:10 and/or a light chain that comprises the amino acid sequence of SEQ ID NO:11. In some embodiments, the anti-HER2 antibody comprises a heavy chain that comprises the amino acid sequence of SEQ ID NO: 9 or SEQ ID NO: 10 and a light chain that comprises the amino acid sequence of SEQ ID NO: 11.

In some embodiments, the anti-HER2 antibody comprises one, two, three, four, five, or six CDR sequences shown in Table 2 below. In some embodiments, the anti-HER2 antibody comprises one, two, or all three CDR sequences from a VH domain or heavy chain sequence shown in Table 2 below and/or one, two, or all three CDR sequences from a VL domain or light chain sequence shown in Table 2 below. In some embodiments, the anti-HER2 antibody comprises a VH domain sequence and/or VL domain sequence shown in Table 2 below. In some embodiments, the anti-HER2 antibody comprises a heavy chain and/or a light chain sequence shown in Table 2 below.

TABLE 2
Anti-HER2 antibody sequences

| Description | Amino acid sequence | SEQ ID NO |
|---|---|---|
| Disitamab | DYYIH | 1 |
| CDR-H1 | | |
| Disitamab | RVNPDHGDSYYNQKFKD | 2 |
| CDR-H2 | | |
| Disitamab | NYLFDH | 3 |
| CDR-H3 | | |
| Disitamab | KASQDVGTAVA | 4 |
| CDR-L1 | | |
| Disitamab | WASIRHT | 5 |
| CDR-L2 | | |
| Disitamab | HQFATYT | 6 |
| CDR-L3 | | |
| Disitamab | EVQLVQSGAEVKKPGATVKISCKVSGYTFTDYYIHWV | 7 |
| VH domain | QQAPGKGLEWMGRVNPDHGDSYYNQKFKDKATITAD | |
| | KSTDTAYMELSSLRSEDTAVYFCARNYLFDHWGQGTL | |
| | VTVSS | |
| Disitamab | DIQMTQSPSSVSASVGDRVTITCKASQDVGTAVAWYQ | 8 |
| VL domain | QKPGKAPKLLIYWASIRHTGVPSRFSGSGSGTDFTLTIS | |
| | SLQPEDFATYYCHQFATYTFGGGTKVEIK | |
| Disitamab | EVQLVQSGAEVKKPGATVKISCKVSGYTFTDYYIHWV | 9 |
| heavy chain | QQAPGKGLEWMGRVNPDHGDSYYNQKFKDKATITAD | |
| sequence 1 | KSTDTAYMELSSLRSEDTAVYFCARNYLFDHWGQGTL | |
| | VTVSSASTKGPSVFPLAPSSKSTSGGTAALGCLVKDYF | |
| | PEPVTVSWNSGALTSGVHTFPAVLQSSGLYSLSSVVTV | |
| | PSSSLGTQTYICNVNHKPSNTKVDKKVEPKSCDKTHTC | |
| | PPCPAPELLGGPSVFLFPPKPKDTLMISRTPEVTCVVVD | |
| | VSHEDPEVKFNWYVDGVEVHNAKTKPREEQYNSTYR | |
| | VVSVLTVLHQDWLNGKEYKCKVSNKALPAPIEKTISK | |
| | AKGQPREPQVYTLPPSREEMTKNQVSLTCLVKGFYPSD | |
| | IAVEWESNGQPENNYKTTPPVLDSDGSFFLYSKLTVDK | |
| | SRWQQGNVFSCSVMHEALHNHYTQKSLSLSPG | |
| Disitamab | EVQLVQSGAEVKKPGATVKISCKVSGYTFTDYYIHWV | 10 |
| heavy chain | QQAPGKGLEWMGRVNPDHGDSYYNQKFKDKATITAD | |
| sequence 2 | KSTDTAYMELSSLRSEDTAVYFCARNYLFDHWGQGTL | |
| | VTVSSASTKGPSVFPLAPSSKSTSGGTAALGCLVKDYF | |
| | PEPVTVSWNSGALTSGVHTFPAVLQSSGLYSLSSVVTV | |
| | PSSSLGTQTYICNVNHKPSNTKVDKKVEPKSCDKTHTC | |
| | PPCPAPELLGGPSVFLFPPKPKDTLMISRTPEVTCVVVD | |
| | VSHEDPEVKFNWYVDGVEVHNAKTKPREEQYNSTYR | |
| | VVSVLTVLHQDWLNGKEYKCKVSNKALPAPIEKTISK | |
| | AKGQPREPQVYTLPPSREEMTKNQVSLTCLVKGFYPSD | |
| | IAVEWESNGQPENNYKTTPPVLDSDGSFFLYSKLTVDK | |
| | SRWQQGNVFSCSVMHEALHNHYTQKSLSLSPGK | |
| Disitamab | DIQMTQSPSSVSASVGDRVTITCKASQDVGTAVAWYQ | 11 |
| light chain | QKPGKAPKLLIYWASIRHTGVPSRFSGSGSGTDFTLTIS | |
| | SLQPEDFATYYCHQFATYTFGGGTKVEIKRTVAAPSVF | |
| | IFPPSDEQLKSGTASVVCLLNNFYPREAKVQWKVDNA | |
| | LQSGNSQESVTEQDSKDSTYSLSSTLTLSKADYEKHKV | |
| | YACEVTHQGLSSPVTKSFNRGEC | |
| Disitamab | ARNYLFDHW | 12 |
| CDR-H3 | | |
| (long) | | |

In some embodiments, the anti-HER2 antibody is disitamab (RC48). See, e.g., U.S. Pat. No. 10,087,260, the contents of which are incorporated herein by reference in its entirety.

In some embodiments, the cytotoxic molecule of an antibody-drug conjugate of the present disclosure comprises a tubulin inhibitor or DNA damaging agent.

In some embodiments, the tubulin inhibitor comprises a dolastatin or derivative thereof, auristatin or derivative thereof, or maytansinoid or derivative thereof. In some embodiments, the tubulin inhibitor comprises monomethyl auristatin E (MMAE), monomethyl auristatin F (MMAF), or auristatin F (AF). In some embodiments, the tubulin inhibitor comprises mertansine (DM1), maytansine (DM3), or ravtansine (DM4). Structures for MMAE and MMAF are provided below. Additional descriptions and examples of tubulin inhibitors may be found, e.g., in Chen, H. et al. (2017) Molecules 22 (8): 1281.

In some embodiments, the DNA damaging agent comprises a calicheamicin, duocarmycin, pyrrolobenzodiazepine (PBD), or SN-38.

In some embodiments, the cytotoxic molecule comprises an amanitin, anthracycline, baccatin, camptothecin, cemadotin, colchicine, colcemid, combretastatin, cryptophycin, discodermolide, docetaxel, doxorubicin, echinomycin, eleutherobin, epothilone, estramustine, lexitropsins, maytansine, methotrexate, netropsin, puromycin, rhizoxins, taxane, tubulysin, or vinca alkaloid.

In some embodiments, the antibody-drug conjugate is represented by formula Ab-(L-U)n, wherein Ab is the anti-HER2 antibody, L is a linker between the cytotoxic molecule and the anti-HER2 antibody, U is the conjugated cytotoxic molecule, and n is an integer from 1 to 8, representing the number of cytotoxic molecules bound to the antibody. For example, in some embodiments, the antibody-drug conjugate used is named RC48-mc-vc-pAB-MMAE, which conforms to the structure of the general formula Ab-(L-U)n, in which RC48 (a humanized anti-HER2 monoclonal antibody) is coupled to MMAE through the linker mc-vc-pAB, and the number of coupling ranges from 1 to 8, including 1, 2, 3, 4, 5, 6, 7, 8 or a combination of antibody-drug conjugates with varying MMAE coupling numbers ranging from 1 to 8. In some embodiments, the number of cytotoxic molecules bound to the antibody is given as an average number of cytotoxic molecules bound to the antibody, e.g., within a given sample, population, or composition. In some embodiments, an average of 4 MMAE molecules are conjugated to the antibody, e.g., via a linker such as mc-vc-pAB.

In some embodiments, the linker is attached to the anti-HER2 antibody via a thiol or amino moiety. In some embodiments, the cytotoxic molecule is conjugated to the antibody through site-directed or undirected conjugation.

In some embodiments, the linker is selected from the group consisting of maleimidocaproyl valine citrulline p-amino-benzyloxy (mc-vc-pAB), maleimidocaproyl (mc), triglycol peptide linker, 3-maleimido-propionic acid, Mal-di-EG-OPFP (perfluorophenyl 3-(2-(2-(3-(2,5-dioxo-2,5-dihydro-1H-pyrrol-1-yl) propanamido) ethoxy) ethoxy) propanoate), Mal-di-EG-Osu (2,5-dioxopyrrolidin-1-yl 3-(2-(2-(2,5-dioxo-2,5-dihydro-1H-pyrrol-1-yl) ethoxy) ethoxy) propanoate), Mal-Tri-EG-Osu (2,5-dioxopyrrolidin-1-yl 3-(2-(2-(2-(2,5-dioxo-2,5-dihydro-1H-pyrrol-1-yl) ethoxy) ethoxy) ethoxy) ethoxypropanoate), Mal-Tetra-EG-Osu (2,5-dioxopyrrolidin-1-yl 1-(2,5-dioxo-2,5-dihydro-1H-pyrrol-1-yl)-3-oxo-7,10,13,16-tetraoxa-4-azanonadecan-19-oate), Br-di-EG-Osu (2,5-dioxopyrrolidin-1-yl 3 (2-(2-(2-bromoacetamido) ethoxy) ethoxy) propanoate), Py-ds-prp-Osu (2-5-dioxopyrrolidin-1-yl 3-(pyridine-2-yldisulfanyl) propanoate), Py-ds-Prp-OPEP (perfluorophenyl 3-(pyridine-2-yldisulfanyl) propanoate), Py-ds-dmBut-Osu (2,5-dioxopyrrolidin-1-yl 4-methyl-4-(pyridine-2-yldisulfanyl) pentanoate, Py-ds-dmBut-OPF (perfluorophenyl 4-methyl-4-(pyridine-2-yldisulfanyl) pentanoate), SMCC (N-succinimidyl 4-(maleimidomethyl)cyclohexanecarboxylate), MBS (3-maleimidobenzoic acid N-hydroxysuccinimide ester), SATA (S—(N-succinimidyl)thioacetate), SPDP ((N-succinimidyl 3-(2-pyridyldithio) propionate), and SMPT ((N-succinimidyloxy carbonyl)-1-methyl-1-(2-pyridyldithio) toluene).

In some embodiments, the linker is a linker described in Table 3 below.

TABLE 3
Exemplary linkers for antibody-drug conjugates.

| Abbreviation | Full name | Feature |
|---|---|---|
| mc | Maleimidocaproyl | Non- |
| | | cleavable |
| mc-vc-pAB | Maleimidocaproyl valine citrulline p-amino-benzyloxy | Cleavable |
| 3-MPA | 3-maleimido-propionic acid | Non- |
| | | cleavable |
| Mal-di-EG- | Perfluorophenyl | Non- |
| OPFP | 3-(2-(2-(3-(2,5-dioxo-2,5-dihydro-1H-pyrrol-1- | cleavable |
| | yl)propanamido)ethoxy) ethoxy)propanoate | |
| Mal-di-EG-Osu | 2,5-dioxopyrrolidin-1-yl | Non- |
| | 3-(2-(2-(2,5-dioxo-2,5-dihydro-1H-pyrrol-1- | cleavable |
| | yl)ethoxy)ethoxy) propanoate | |
| Mal-Tri-EG-Osu | 2,5-dioxopyrrolidin-1-yl | Non- |
| | 3-(2-(2-(2-(2,5-dioxo-2,5-dihydro-1H-pyrrol-1- | cleavable |
| | yl)ethoxy)ethoxy)ethoxy) propanoate | |
| Mal-Tetra-EG- | 2,5-dioxopyrrolidin-1-yl | Non- |
| Osu | 1-(2,5-dioxo-2,5-dihydro-1H-pyrrol-1-yl)-3-oxo- | cleavable |
| | 7,10,13,16-tetraoxa-4- | |
| | azanonadecan-19-oate | |
| Br-di-EG-Osu | 2,5-dioxopyrrolidin-1-yl | Non- |
| | 3-(2-(2-(2-bromoacetamido)ethoxy)ethoxy)propanoate | cleavable |
| Py-ds-prp-Osu | 2,5-dioxopyrrolidin-1-yl 3-(23escry23e-2- | Reducible |
| | yldisulfanyl)propanoate | |
| Py-ds-Prp-OPFP | Perfluorophenyl 3-(23escry23e-2- | Reducible |
| | yldisulfanyl)propanoate | |
| Py-ds-dmBut- | 2,5-dioxopyrrolidin-1-yl 4-methyl-4-(23escry23e-2- | Reducible |
| Osu | yldisulfanyl) | |
| | pentanoate | |
| Py-ds-dmBut- | Perfluorophenyl 4-methyl-4-(23escry23e-2- | Reducible |
| OPF | yldisulfanyl)pentanoate | |
| SMCC | N-succinimidyl 4-(maleimidomethyl) | Non- |
| | cyclohexanecarboxylate | cleavable |
| MBS | 3-maleimidobenzoic acid N-hydroxysuccinimide ester | Non- |
| | | cleavable |
| SATA | S-(N-succinimidyl) thioacetate | Non- |
| | | cleavable |
| SPDP | N-succinimidyl 3-(2-pyridyldithio)propionate | Reducible |
| SMPT | (N-succinimidyl carbonyl)-1-methyl-1-(2- | Reducible |
| | pyridyldithio) toluene | |

In some embodiments, the anti-HER2 antibody-drug conjugate is disitamab vedotin (DV; also referred to as RC48-ADC). Disitamab vedotin (DV hereafter) is an antibody-drug conjugate (ADC) that targets cancers expressing HER2, an oncogenic growth factor receptor which promotes cell proliferation and survival. DV consists of an anti-HER2 monoclonal antibody disitamab (RC48) conjugated with an average of 4 molecules of the tubulin-disrupting anti-mitotic agent monomethyl auristatin E (MMAE) via a cleavable peptide linker. See, e.g., U.S. Pat. No. 10,087,260 and U.S. PG Pub Nos. 2020/0289663 and 2021/0154314, the contents of which are incorporated herein by reference in their entirety. DV has multimodal antitumor mechanisms of action that include direct cytotoxicity of HER2-expressing cancer cells and bystander effect based-cytotoxicity of neighboring cells, both of which are mediated by the intracellular release of MMAE within the targeted cell. Released MMAE can induce immunogenic cell death (ICD), which promotes immune cell recruitment to the tumor. In addition, DV stimulates Fc-gamma receptor mediated antibody-dependent cellular cytotoxicity (ADCC), which can lead to target cell death. DV also inhibits HER2-activated downstream signaling pathways, further blocking cellular growth and proliferation.

### Cancers to be Treated

In some embodiments, the cancer is head and neck squamous cell carcinoma (HNSCC), ovarian cancer, non-small cell lung cancer (NSCLC), or endometrial cancer. In some embodiments, the cancer is head and neck squamous cell carcinoma (HNSCC), ovarian cancer, or endometrial cancer. In some embodiments, the cancer is non-small cell lung cancer (NSCLC). In some embodiments, the cancer is head and neck squamous cell carcinoma (HNSCC). In some embodiments, the cancer is ovarian cancer. In some embodiments, the cancer is non-small cell lung cancer (NSCLC). In some embodiments, the cancer is endometrial cancer. In some embodiments, the cancer is locally advanced or metastatic (LA/m).

In some embodiments, cells of the cancer express HER2. In some embodiments, the cancer is a HER2-positive (HER2+) cancer. In some embodiments, cells of the cancer exhibit HER2 gene amplification. In some embodiments, cells of the cancer overexpress HER2, e.g., on their cell surface. Overexpression and/or amplification of HER2 is seen in many malignancies including breast, gastric, ovarian, pancreatic, colorectal, and endometrial cancer (Neve, R. M. et al. (2001) Ann Oncol 12(Suppl1):S9-S13; Menard, S. et al. (2003) Oncogene 22(42):6570-6578; Moasser, M. M. (2007) Oncogene 26(45):6469-6487; Iqbal, N. et al. (2014) Mol Biol Int 2014:852748). In some embodiments, cells of the cancer express HER2, wherein the cells have an IHC score of IHC≥1 and/or are ISH positive.

In some embodiments, a sample obtained from the individual comprises cancer cells that express HER2 on their cell surface, as measured by an immunohistochemistry (IHC) assay. IHC assays for HER2 involve semi-quantitative measurements of HER2 cellular expression/overexpression. These are typically done by staining a sample including cancer cells using a primary anti-HER2 antibody, followed by visualization using a labeled secondary antibody and subsequent analysis of HER2 expression, e.g., membrane expression on the surface. Variables factoring into the IHC score (given on a scale from 0 to 3+) can include intensity of staining, location and/or completeness of staining (e.g., surface/membrane expression), and proportion of tumor cells exhibiting staining. Methods and criteria for determining HER2 status by IHC are known in the art and can be found, e.g., in Wolff, A. C. et al. (2013) J Clin Oncol 31 (31): 3997-4013 and Wolff, A. C. et al. (2018) J Clin Oncol 36 (20): 2105-2122. See also the HercepTest™ semi-quantitative IHC test kit.

Evaluation of HER2 by IHC May Involve One or More, or all, of the Steps of:
1. The entire section is first observed under low magnification to determine whether the staining is satisfactory and whether there is heterogeneity in HER2 expression;
2. Quality control slides are read when evaluating; cytoplasmic and nuclear staining should be negligible, and normal epithelium should not show strong cell membrane staining;
3. Tissue margins and poorly prepared (e.g., obviously extruded) cancer tissue is ignored during evaluation.
4. If the tumor has obvious heterogeneity, the percentage of each scoring level is indicated separately when interpreting.
5. If invasive cancer is the object during evaluation, it is indicated separately if the non-invasive cancer part has overexpressed HER2 (2+ or 3+).
6. If multiple blocks or sections are detected, results are reported separately.

In some embodiments, a sample obtained from the individual comprises cancer cells that exhibit HER2 gene amplification, as measured by an in situ hybridization (ISH) assay (ISH-positive). In some embodiments, a sample obtained from the individual comprises cancer cells that do not exhibit HER2 gene amplification, as measured by an in situ hybridization (ISH) assay (ISH-negative). ISH assays for HER2 gene amplification typically involve measurement of level of hybridization to a HER2-specific probe using microscopy. In some embodiments, cells are stained with dual probes: a HER2-specific probe, and a control probe (hybridizing to, e.g., chromosome 17 or CEP17), such that the ratio of HER2:control signal is indicative of HER2 copy number and/or amplification. Variables factoring into the ISH status can include number of signal copies of HER2, ratio of HER2:control copy number, and formation of HER2 clusters. Methods and criteria for determining HER2 amplification status by ISH are known in the art and can be found, e.g., in Wolff, A. C. et al. (2013) J Clin Oncol 31 (31): 3997-4013 and Wolff, A. C. et al. (2018) J Clin Oncol 36 (20): 2105-2122. Detection of HER2 may be performed by FISH, e.g., using one or more, or all, of the following steps:
(1) Selecting a representative wax block of tumor tissue. Section by professional and technical personnel, the section is complete, smooth, of uniform thickness, without affecting the diagnosis of knife mark wrinkles. (Tissue containing calcified particles and other uncontrollable factors are excluded), section thickness: 4-5 μm;
(2) Tissue section pretreatment using either of the following methods:
Method 1 (Manual operation):
   a) Immerse in xylene and dewaxed twice, 15 minutes each time, and then immerse in 100% ethanol for 5 minutes at room temperature,
   b) Rehydrate in 100% ethanol, 85% ethanol and 70% ethanol for 2 minutes respectively at room temperature, then immerse in deionized water at room temperature for 3 minutes,
   c) Treatment with 90˜93° C. deionized water for 20 minutes,
   d) 1 ml gastric enzyme storage solution (200 mg/mL) is dissolved in 200 ml 0.01 MHCL to obtain gastric enzyme working solution (1 mg/ml); Soak the tissue section in gastric enzyme working solution and incubate at 37° C. for 15-30 minutes (the time depends on the thickness of the tissue, generally about 20 minutes),
   e) After digestion by gastric enzymes, then rinse in deionized water for 5 minutes,
   f) Dehydrate respectively in 70% ethanol, 85% ethanol and 100% ethanol for 2 minutes at room temperature,
   g) After drying, then perform the following hybrid denaturation.
Method 2 (Fully automatic):
   a) Soak in xylene for dewaxing twice at room temperature for 15 minutes each, and then immerse in 100% ethanol twice for 5 minutes each,
   b) Dry tissue section at room temperature,
   c) Initialize the system and select program, fill the reagent according to the instrument algorithm,
   d) Place the dry slides tissue face upward on the glass shelf, put it in the reaction tank, cover the reaction tank cover, close the machine cover, and run the selected program;
(3) Hybridization apparatus denatured hybridization using the following steps:
   a) Drop 10 μL probe mixture into the slide hybridization area, immediately cover the slide and seal the edge with rubber glue,
   b) Prepare hybridization machine, covariance condition: 75° C., 5 minutes, hybridization condition: 37° C., 16 h; (be careful to maintain humidity in hybridization instrument);
(4) Glass slide rinsing (need to avoid light operation) using the following steps:
   a) Carefully remove the cover glass slide, place the glass slide in a solution of 0.3% NP-40/2×SSC at 73° C., shake for 1˜3 seconds, wash for 2 minutes,
   b) Rinse at room temperature in 70% ethanol for 3 minutes;
(5) Counterstaining using the following steps:
   a) Naturally dried glass slides in dark;
   b) Drop 10 μL DAPI at the hybridization site and immediately cover the cover glass. Put in the dark for 10 to 20 minutes, then observe the glass slides under fluorescence microscope with appropriate filter group.

Assessment of HER2, e.g., in a FISH section, for example, generated as described above, may be performed using any suitable method known in the art. For example, using one or more, or all of the following steps:
(a) Observe whole FISH section under low magnification to preliminarily determine the test quality (such as the normal cell signals of normal tissues in the specimen) and whether there is heterogeneity in HER2 amplification;
(b) Find at least 2 areas of invasive cancer and count at least 20 invasive cancer cells. FISH is not suitable for microinvasive nidus with too few cells;
(c) IHC sections can be used to determine the areas of invasive cancer that may be amplified; and
(d) Observe HER2 and CEP17 signals through a specific channel filter under high magnification (60× or 100× objective), and calculate the signal count and ratio.

In some embodiments, the sample is a biopsy sample, e.g., from a core needle biopsy. In some embodiments, the sample is from an incisional or excisional surgical procedure. In some embodiments, the sample is a formalin-fixed paraffin embedded (FFPE) tissue block or sample, e.g., with corresponding H & E stain. In some embodiments, the sample comprises unstained slides sectioned from a tissue block, e.g., FFPE tissue block. In some embodiments, the sample is from a primary tumor or metastasis, e.g., from the chest wall, regional lymph node, or a distant organ. In some embodiments, the same sample is used for IHC and ISH assays. In some embodiments, different samples from the same individual are used for IHC and ISH assays.

In some embodiments, the cancer is a HER2-low cancer. In some embodiments, a sample obtained from the individual (e.g., from the cancer of the individual) comprises cancer cells that express HER2 on their cell surface at a level of IHC1+, as measured by IHC assay (IHC1+). In some embodiments, a sample obtained from the individual (e.g., from the cancer of the individual) comprises cancer cells that express HER2 on their cell surface at a level of IHC2+, as measured by IHC assay (IHC2+); and a sample obtained from the individual comprises cancer cells that do not exhibit HER2 gene amplification, as measured by ISH assay (IHC2+/ISH-negative). In some embodiments, a sample obtained from the individual (e.g., from the cancer of the individual) is IHC1+. In some embodiments, a sample obtained from the individual (e.g., from the cancer of the individual) is IHC2+/ISH-negative.

In some embodiments, the cancer is a HER2-positive cancer. In some embodiments, a sample obtained from the individual (e.g., from the cancer of the individual) comprises cancer cells that express HER2 on their cell surface at a level of IHC3+, as measured by IHC assay (IHC3+). In some embodiments, a sample obtained from the individual (e.g., from the cancer of the individual) comprises cancer cells that express HER2 on their cell surface at a level of IHC2+, as measured by IHC assay (IHC2+); and a sample obtained from the individual (e.g., from the cancer of the individual) comprises cancer cells that exhibit HER2 gene amplification, as measured by ISH assay (IHC2+/ISH-positive). In some embodiments, a sample obtained from the individual (e.g., from the cancer of the individual) is IHC3+. In some embodiments, a sample obtained from the individual (e.g., from the cancer of the individual) is IHC3+/ISH+.

In some embodiments, the cancer is HER2+ (IHC≥1+) locally-advanced, unresectable or metastatic (LA/m) head and neck squamous cell carcinoma (HNSCC), non-small cell lung cancer (NSCLC), ovarian cancer, or endometrial cancer. In some embodiments, the cancer is HER2+ (IHC≥1+) LA/m HNSCC. In some embodiments, the cancer is HER2+ (IHC≥1+) LA/m NSCLC. In some embodiments, the cancer is HER2+ (IHC≥1+) LA/m ovarian cancer. In some embodiments, the cancer is HER2+ (IHC≥1+) LA/m endometrial cancer.

In some embodiments, the cancer expresses a low level of HER2, but is classified as a HER2-zero cancer. In some embodiments, a sample obtained from the individual (e.g., from the cancer of the individual) comprises cancer cells that express HER2 on their cell surface at a level of IHC 0, as measured by IHC assay (IHC 0). In some embodiments, a sample obtained from the individual (e.g., from the cancer of the individual) is IHC 0. In some embodiments, the cancer is HER2-zero (IHC 0) locally-advanced, unresectable or metastatic (LA/m) head and neck squamous cell carcinoma (HNSCC), non-small cell lung cancer (NSCLC), ovarian cancer, or endometrial cancer. In some embodiments, the cancer is HER2-zero (IHC 0) LA/m HNSCC. In some embodiments, the cancer is HER2-zero (IHC 0) LA/m NSCLC. In some embodiments, the cancer is HER2-zero (IHC 0) LA/m ovarian cancer. In some embodiments, the cancer is HER2-zero (IHC 0) LA/m endometrial cancer.

### Individuals to be Treated

In some embodiments, e.g., prior to treatment according to the methods disclosed herein, the individual has been previously treated for the cancer. In some embodiments, the cancer has previously been treated. For example, in some embodiments, the administration of the antibody-drug conjugate according to the methods of the present disclosure is a second-line (2 L) treatment or a third-line or higher (3 L+) treatment. In some embodiments, the patient has been previously treated with a HER2 directed antibody-drug conjugate.

In some embodiments, e.g., prior to administration of the antibody-drug conjugate, the individual has progressed during and/or after ≥1 prior line of systemic therapy for LA/m disease. In some embodiments, e.g., prior to administration of the antibody-drug conjugate, the individual has progressed during and/or after the most recent line of systemic therapy. In some embodiments, prior to administration of the antibody-drug conjugate, the individual is intolerant of the most recent line of systemic therapy. In some embodiments, e.g., prior to administration of the antibody-drug conjugate, the individual has progressed during or after ≥1 prior line of systemic therapy for LA/m disease. In some embodiments, e.g., prior to administration of the antibody-drug conjugate, the individual is intolerant of the most recent line of systemic therapy.

In some embodiments, e.g., prior to treatment according to the methods disclosed herein, the individual is at least 18 years of age. In some embodiments, e.g., prior to treatment according to the methods disclosed herein, the individual is at least 18 years of age or considered an adult in the jurisdiction in which the study is taking place at the time of consent. In some embodiments, e.g., prior to treatment according to the methods disclosed herein, the individual has measurable disease according to RECIST v1.1 guidelines.

In some embodiments, e.g., prior to treatment according to the methods disclosed herein, the individual has disease (e.g., cancer) demonstrating HER2 expression 1+, 2+, or 3+, as determined by local IHC testing on a fresh or archival tumor tissue (IHC1+, IHC2+, IHC3+, respectively). In some embodiments, the gastric cancer scoring algorithm is used to determine IHC score.

In some embodiments, e.g., prior to treatment according to the methods disclosed herein, the individual has an Eastern Cooperative Oncology Group (ECOG) Performance Status score of 0 or 1. In some embodiments, e.g., prior to treatment according to the methods disclosed herein, the individual has one, some, or all of the following lab values: ALT and AST≤3× upper limit of normal (ULN) without liver metastases or ≤5×ULN with liver metastases; total bilirubin≤1.5×ULN; direct bilirubin≤1.5×ULN and normal AST and ALT for subjects with known history of Gilbert's syndrome; absolute neutrophil count (ANC)≥1.5×10⁹/L; platelet count ≥100×10⁹/L; hemoglobin ≥9.0 g/dL (≥90 g/L); estimated glomerular filtration rate (GFR)≥30 mL/min/1.73 m², as measured by the Cockcroft-Gault formula, Modification of Diet in Renal Disease [MDRD] or 24-hour urine. In embodiments wherein the individual was transfused with any blood product prior to treatment according to the methods disclosed herein, transfusion took place ≥14 days prior to start of therapy to establish adequate hematologic parameters independent from transfusion support. In some embodiments, e.g., prior to treatment according to the methods disclosed herein, the individual has a left ventricular ejection fraction (LVEF)≥50% as assessed by echocardiogram or MUGA documented within ≤3 months prior to first dose of study treatment. In some embodiments, e.g., prior to treatment according to the methods disclosed herein, the individual has a Fridericia's corrected QT interval (QTcF)<470 ms.

In some embodiments, e.g., for individuals with HNSCC, the individual has pathologically-documented squamous cell carcinoma of the head and neck with primary tumor site arising from the oral cavity, oropharynx, hypopharynx, and larynx. In some embodiments, the cancer is HNSCC with a primary tumor site arising from the oral cavity, oropharynx, hypopharynx, or larynx. In some embodiments, e.g., for individuals with HNSCC, the tumor site of the cancer does not arise from the nasopharynx. In some embodiments, e.g., for individuals with HNSCC, the cancer is unresectable locally recurrent or metastatic stage. In some embodiments, the individual has received prior anti-PD(L)1 therapy. In some embodiments, e.g., for individuals with HNSCC, one or more of the following apply:
Disease progression after treatment with a platinum-based therapy:
   For recurrent disease not amenable to curative treatment or metastatic disease; or
   Experienced recurrence/progression within 6 months of last dose of platinum therapy administered as part of multimodal therapy in the curative setting.
No more than 1 line of cytotoxic chemotherapy for advanced disease.

In some embodiments, e.g., for individuals with NSCLC, the individual has a known EGFR, ALK, ROS or other actionable mutation and has received targeted therapy. In some embodiments, the cancer is NSCLC with a mutation in EGFR, ALK, or ROS, and the individual has received prior targeted therapy. In some embodiments, e.g., for individuals with NSCLC, the individual does not have small cell carcinoma. In some embodiments, e.g., for individuals with NSCLC, the individual does not have one or more known HER2 mutations. In other embodiments, the individual has one or more known HER2 mutations. In some embodiments, e.g., prior to administration of the antibody-drug conjugate, the individual (a) progressed during or after a platinum-based therapy or, within 6 months of platinum-based adjuvant, neoadjuvant, or concomitant chemoradiotherapy for early or locally-advanced stage disease; or (b) received prior anti-PD(L)1 therapy.

In some embodiments, e.g., for individuals with NSCLC, one or more of the following apply:
Subject has pathologically documented NSCLC.
Subject has unresectable locally-advanced or metastatic stage disease.
Subjects has progressed during or after a platinum-based therapy or, within 6 months of platinum-based adjuvant, neoadjuvant, or concomitant chemoradiotherapy for early or locally-advanced stage disease.
Subject has received prior anti-PD(L)1 therapy, unless contraindicated.
No more than 2 prior lines of cytotoxic chemotherapy for advanced disease.

In some embodiments, e.g., for individuals with ovarian cancer, the cancer is a pathologically documented epithelial cancer of ovarian, fallopian tube, or peritoneal origin. In some embodiments, the cancer is ovarian cancer comprising epithelial cancers of ovarian, fallopian tube, or peritoneal origin. In some embodiments, e.g., for individuals with ovarian cancer, the individual has a known BRCA mutation and has received targeted therapy with a PARP inhibitor. In some embodiments, the cancer is ovarian cancer with a BRCA mutation, and the individual has received prior treatment with a PARP inhibitor. In some embodiments, e.g., for individuals with ovarian cancer, one or more of the following apply:
Subject has platinum resistant disease (6 months or less between the completion of platinum-based treatment and identification of recurrence).
No more than 4 lines of prior cytotoxic chemotherapies for advanced disease
Subject has received prior anti-PD(L)1 therapy.

In some embodiments, e.g., for individuals with endometrial cancer, the individual has pathologically documented adenocarcinoma of the endometrium. In some embodiments, the cancer is adenocarcinoma of the endometrium. In some embodiments, e.g., for individuals with endometrial cancer, one or more of the following apply:
Subject has unresectable locally-advanced or metastatic stage disease.
Subject has relapsed/progressed after at least one prior platinum-based chemotherapy for recurrent, metastatic or primary unresectable disease. Subjects who experience disease progression less than 6 months after completion of adjuvant or neoadjuvant platinum-based chemotherapy are also eligible
No more than 3 lines of prior cytotoxic chemotherapies for advanced disease
Subject has received prior anti-PD(L)1 therapy.

In some embodiments, the individual has one or more known HER2 mutations.

In some embodiments, the individual is a human.

### Administration

In some embodiments, the antibody-drug conjugate is administered to the individual at a dose of 1.5 mg/kg. In some embodiments, the antibody-drug conjugate is administered to the individual at a maximum dose of 150 mg. In some embodiments, the antibody-drug conjugate is administered intravenously to the individual.

The concentration of a protein-based drug substance can be estimated using various methods that typically reference a comparative standard. Methods and reference standards evolve over time to be more precise and accurate in determining protein concentrations. In some embodiments, the dose is measured using the disitamab vedotin-based Extinction Coefficient (EC) method. The disitamab vedotin-based Extinction Coefficient (EC) method is a UV spectrophotometric method for determining the protein concentration using the sample's absorbance and an extinction coefficient (EC). Exemplary protocols are provided in Example 2. In some embodiments, the antibody-drug conjugate is administered to the individual at a dose of 1.5 mg/kg, measured using the disitamab vedotin-based EC method. In other embodiments, the dose contains the same mole-quantity of the drug as 1.5 mg/kg measured using the disitamab vedotin-based EC method, but is measured using another method and therefore expressed differently, for example, as 2.0 mg/kg dose as measured using a bovine-serum albumin (BSA)-based EC method. An exemplary protocol of the BSA-based EC method is provided in Example 5.

In some embodiments, the antibody-drug conjugate is administered to the individual every 2 weeks or every 14 days (Q2W). In some embodiments, the antibody-drug conjugate is disitamab vedotin, and the method comprises intravenously administering disitamab vedotin to the individual at 1.5 mg/kg with a maximum dose of 150 mg every two weeks or every 14 days (Q2W). In some embodiments, the antibody-drug conjugate is administered to the individual every 11 to 17 days. In some embodiments, the antibody-drug conjugate is administered to the individual every 11 days. In some embodiments, the antibody-drug conjugate is administered to the individual every 12 days. In some embodiments, the antibody-drug conjugate is administered to the individual every 13 days. In some embodiments, the antibody-drug conjugate is administered to the individual every 14 days. In some embodiments, the antibody-drug conjugate is administered to the individual every 15 days. In some embodiments, the antibody-drug conjugate is administered to the individual every 16 days. In some embodiments, the antibody-drug conjugate is administered to the individual every 17 days. In some embodiments, the antibody-drug conjugate is disitamab vedotin, and the method comprises intravenously administering disitamab vedotin to the individual at 1.5 mg/kg with a maximum dose of 150 mg every 11 to 17 days. In some embodiments, the antibody-drug conjugate is disitamab vedotin, and the method comprises intravenously administering disitamab vedotin to the individual at 1.5 mg/kg with a maximum dose of 150 mg every 11 days. In some embodiments, the antibody-drug conjugate is disitamab vedotin, and the method comprises intravenously administering disitamab vedotin to the individual at 1.5 mg/kg with a maximum dose of 150 mg every 12 days. In some embodiments, the antibody-drug conjugate is disitamab vedotin, and the method comprises intravenously administering disitamab vedotin to the individual at 1.5 mg/kg with a maximum dose of 150 mg every 13 days. In some embodiments, the antibody-drug conjugate is disitamab vedotin, and the method comprises intravenously administering disitamab vedotin to the individual at 1.5 mg/kg with a maximum dose of 150 mg every 14 days. In some embodiments, the antibody-drug conjugate is disitamab vedotin, and the method comprises intravenously administering disitamab vedotin to the individual at 1.5 mg/kg with a maximum dose of 150 mg every 15 days. In some embodiments, the antibody-drug conjugate is disitamab vedotin, and the method comprises intravenously administering disitamab vedotin to the individual at 1.5 mg/kg with a maximum dose of 150 mg every 16 days. In some embodiments, the antibody-drug conjugate is disitamab vedotin, and the method comprises intravenously administering disitamab vedotin to the individual at 1.5 mg/kg with a maximum dose of 150 mg every 17 days. In some embodiments, the dose is measured using the disitamab vedotin-based Extinction Coefficient (EC) method.

In some embodiments, the administration of the antibody-drug conjugate according to the methods of the present disclosure is a second-line (2 L) treatment. In some embodiments, the administration of the antibody-drug conjugate according to the methods of the present disclosure is a third-line or higher (3 L+) treatment.

In some embodiments, administration of the antibody-drug conjugate results in a complete response (CR) or partial response (PR) in the individual. In some embodiments, administration of the antibody-drug conjugate results in a complete response (CR) or partial response (PR) in the individual within about 3 years. In some embodiments, response to treatment according to the methods disclosed herein is evaluated using RECIST v1.1 criteria. Exemplary criteria are provided below.

Response Evaluation Criteria for Solid Tumors (RECIST) Version 1.1
(Eisenhauer, E.A. et al. (2009) Eur J Cancer 45(2): 228-247)

| Term | Definition |
|---|---|
| Complete Response | Disappearance of all target lesions. Any pathological lymph |
| (CR) | nodes (whether target or non-target) must have reduction in short |
| | axis to <10 mm |
| Partial Response (PR) | A ≥30% decrease in the sum of diameters of target lesions |
| | (longest for non-nodal target lesions and the short axes for nodal |
| | target lesions), taking as reference the baseline sum diameters |
| Stable Disease (SD) | Neither sufficient shrinkage to qualify for PR nor sufficient |
| | increase to qualify for PD, taking as reference the smallest sum |
| | diameters while on study |
| Progressive Disease | A ≥20% relative increase in the sum of diameters of target |
| (PD) | lesions, taking as reference the smallest sum on study (including |
| | baseline sum if that is the smallest on study). In addition, the sum |
| | must also demonstrate an absolute increase ≥5 mm |
| | The appearance of 1 or more new lesions is also considered |
| | progression |

In some embodiments, the individual is a human.

### EXAMPLES

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### Example 1: Phase 2 Basket Study of Disitamab Vedotin in Adult Subjects with Previously Treated, Locally-Advanced Unresectable or Metastatic Solid Tumors that Express HER2

Patients with locally-advanced, unresectable or metastatic (LA/m) head and neck squamous cell carcinoma (HNSCC), non-small cell lung cancer (NSCLC), ovarian cancer, and endometrial cancer have limited treatment options after front line therapy. Second line and subsequent treatments often result in low response rates and modest duration of disease control. In addition, human epidermal growth factor receptor 2 (HER2) is expressed in a significant proportion of these tumors but therapies targeting HER2 for these tumors have not been developed. There is an urgent need for novel therapies in the late-line setting that can provide clinical benefit.

### Design

This example describes a multi-cohort, multicenter, international, open-label Phase 2 study designed to assess the activity, safety, and tolerability of disitamab vedotin monotherapy for the treatment of subjects with previously treated, LA/m solid tumors with HER2 expression defined by IHC level 1+, 2+, and 3+.

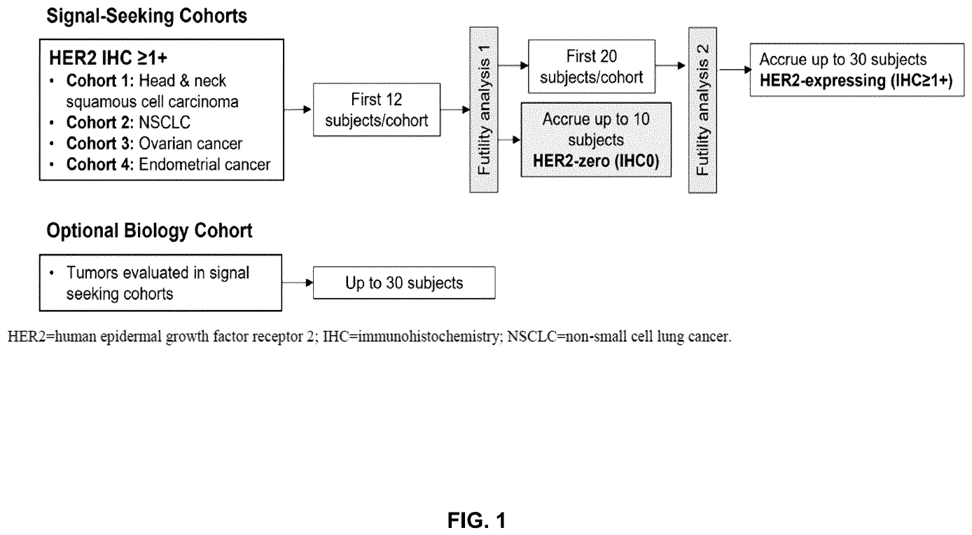

Subjects are enrolled into separate cohorts based on tumor type (see FIG. 1). There are 4 tumor specific cohorts: HNSCC (Cohort 1), NSCLC (Cohort 2), ovarian cancer (Cohort 3), and endometrial cancer (Cohort 4), with approximately 40 subjects in each cohort. Each cohort, up to approximately 30 subjects, whose disease displays HER2 expression of IHC≥1+, are enrolled in cohorts 1 through 4. An additional 10 subjects in each cohort with HER2 IHC 0 are enrolled if a cohort passes the interim futility analysis.

In general, subjects must have progressed during or after ≥1 prior line of systemic therapy for LA/m disease and must have progressed during or after, or be intolerant of, the most recent line of systemic therapy (see section on specific inclusion criteria of each cohort). Subjects must have measurable disease, Eastern Cooperative Oncology Group (ECOG) performance status 0 to 1, and adequate baseline hepatic, renal, and hematologic function. Subjects must not have received prior HER2-directed ADC therapy, or have clinically significant cardiopulmonary disease, chronic liver disease, or uncontrolled central nervous system (CNS) metastases.

Study treatment is composed of disitamab vedotin 1.5 mg/kg Q2W (dose is calculated using the disitamab vedotin-based EC method; i.e., the protein concentration is determined using the sample's absorbance and an extinction coefficient (EC) through a UV spectrophotometric method) until disease progression or unacceptable toxicity, pregnancy, death, withdrawal of consent, or termination of the study by the sponsor.

Two interim futility analyses are performed separately in each HER2 expressing (IHC≥1+) cohort (Cohorts 1-4) after approximately 12 and 20 subjects of given cohort have completed at least 2 post baseline tumor assessments on study or had disease progression, respectively. At the first futility analysis, if there is at least 1 out of 12 subjects with observed Response Evaluation Criteria in Solid Tumours (RECIST) response (CR or PR) in a given cohort, this cohort continues enrollment, and additional 10 subjects with HER2 IHC 0 are treated with disitamab vedotin in this cohort to explore disitamab vedotin activity in this population. If there are 0 out of 12 subjects with observed RECIST response (CR or PR) in given cohort, this cohort discontinues further enrollment. At the second futility analysis, if there are fewer than 3 responses out of 20 subjects in one cohort, this cohort is stopped early. Enrollment is not held during either interim futility analysis.

All subjects undergo serial radiographic assessments to assess tumor response according to RECIST version 1.1 (v1.1) using computed tomography (CT) scan of the chest, CT scan or magnetic resonance imaging (MRI) with contrast of the abdomen, pelvis, and any other non-brain or non-bone sites as clinically indicated. MRI scan (alternatively CT scan) of the brain (with contrast) should be performed in subjects who have a history of CNS metastasis or signs/symptoms of CNS metastasis at screening/baseline.

For all cohorts, disease assessments occur at screening, every 6 weeks ([Q6W]±7 days) up to 72 weeks and every 12 weeks (±14 days) thereafter until occurrence of disease progression, withdrawal of consent, death, or study closure, whichever occurs first. The same imaging modality used at screening should be used throughout the study.

The severity of AEs and laboratory abnormalities is graded using the National Cancer Institute Common Terminology Criteria for AEs (NCI CTCAE), version 5.0. Safety is evaluated by monitoring of AEs, serious adverse events (SAEs), vital signs, physical examinations, laboratory tests (complete blood count [CBC] with differential, blood chemistry panel, and lipid panel), concomitant medications, pregnancy testing, and echocardiogram or multigated acquisition (MUGA)-scan.

Disitamab vedotin PK and antidrug antibody (ADA) sampling is undertaken in all subjects throughout study treatment and at the end of treatment (EOT) visit. Blood samples are obtained in all subjects at protocol-specified timepoints to assess PK.

To further characterize the pharmacodynamics activity of disitamab vedotin, up to approximately 30 additional subjects who consent to research biopsies may be eligible to enroll into a biology cohort. Subjects in the biology cohort are asked to provide pre and post treatment tumor samples to characterize the clinical MOA and correlates of sensitivity or resistance to disitamab vedotin. Approximately 30 subjects with LA/m HER2 expressing HNSCC, NSCLC, ovarian cancer, or endometrial cancer are enrolled in the Biology Cohort. This cohort may be opened at the discretion of the sponsor.

### Treatment

A schema for the study is shown in FIG. 1. All interventions administered in this study are outlined in Table 4.

TABLE 4
Study Interventions

| | | Formulation/ | | | | | Packing |
|---|---|---|---|---|---|---|---|
| | Intervention | Route of | Dose/ | | | | and |
| Intervention | Type/Use | Administration | Potency | Frequency | Schedule | Sourcing | Labeling |
| Disitamab | IMP- | Vial/IV | 1.5 | Q2W | Day 1 | Provided | Packaged |
| vedotin | Biologic/ | Infusion | mg/kg^{a} | | (±3 | centrally | from |
| | Experimental | | Infusion | | days) | by the | clinical |
| | | | time | | every | sponsor | supply |
| | | | approximately | | 14 | | in cartons |
| | | | 60 minutes^{b} | | days^{c} | | of |
| | | | | | | | 6 (45 mg |
| | | | | | | | labelled) |
| | | | | | | | vials. |
| | | | | | | | Boxes |
| | | | | | | | and vials |
| | | | | | | | are |
| | | | | | | | labeled |
| | | | | | | | as per |
| | | | | | | | country |
| | | | | | | | requirements. |

EC = extinction coefficient; IMP = investigational medicinal product; IV = intravenous; Q2W = once every 2 weeks.
^{a}Calculated using the disitamab vedotin-based EC method.
^{b}Infusion time should be approximately 60 minutes if the initial infusion rate is adequately tolerated with no infusion-related reactions ≥ Grade 1. Infusion should be no shorter than 30 minutes, and up to 90 minutes.
^{c}At minimum, there must be 11 days between 2 disitamab vedotin doses.

Subjects are enrolled into separate cohorts based on tumor type. There are 4 tumor specific cohorts: HNSCC (Cohort 1), NSCLC (Cohort 2), ovarian cancer (Cohort 3), and endometrial cancer (Cohort 4), with approximately 40 subjects in each cohort. Approximately 30 subjects are enrolled in each cohort of Cohorts 1 through 4, whose disease exhibit HER2 expression of IHC≥1+. An additional 10 subjects in each cohort with HER2 IHC 0 are enrolled if a cohort passes the first interim futility analysis.

In general, subjects must have progressed during or after ≥1 prior line of systemic therapy for LA/m disease and must have progressed during or after, or be intolerant of, the most recent line of systemic therapy. Subjects must have measurable disease, Eastern Cooperative Oncology Group (ECOG) performance status 0 to 1, and adequate baseline hepatic, renal, and hematologic function. Subjects must not have received prior HER2-directed ADC therapy, or have clinically significant cardiopulmonary disease, chronic liver disease, or uncontrolled central nervous system (CNS) metastases. Study treatment is composed of disitamab vedotin 1.5 mg/kg every 2 weeks. Disease assessments are performed according to Response Evaluation in Solid Tumors version 1.1 (RECIST v1.1).

Two interim futility analyses are performed in each HER2 expressing (IHC≥1+) cohort (Cohorts 1-4) after approximately 12 and 20 subjects of a given cohort have completed at least 2 post baseline tumor assessments on study or had disease progression, respectively. In each cohort, if the first futility analysis is passed, an additional 10 subjects with HER2 IHC 0 may be enrolled. A Biology Cohort may be opened at the discretion of the sponsor and enroll approximately 30 previously treated subjects with LA/m HER2 expressing HNSCC, NSCLC, ovarian cancer, or endometrial cancer.

### Objectives and Endpoints

In this study, the primary objective is to evaluate the antitumor activity of disitamab vedotin in subjects with previously treated, locally-advanced unresectable or metastatic (LA/m) HER2 expressing solid tumors. The endpoint for this objective is confirmed objective response rate (ORR) defined as complete response (CR) or partial response (PR) using the Response Evaluation in Solid Tumors version 1.1 (RECIST v1.1) as assessed by the investigator.

A secondary objective is to evaluate the safety and tolerability profile of disitamab vedotin. Endpoints include:

Type, incidence, severity, seriousness, and relatedness of AEs including AEs of special interest (AESIs)
Type, incidence, and severity of laboratory abnormalities as well as significant changes from baseline
Frequency of treatment interruptions, dose reductions and treatment discontinuations due to AEs

Another secondary objective is to assess other measures of antitumor activity of disitamab vedotin per investigator assessment by other clinically relevant measures. Endpoints include:
Confirmed disease control rate (DCR) defined as CR, PR, or stable disease (SD) using RECIST v1.1 as assessed by the investigator.
Duration of response (DOR) defined as the amount of time subjects with CR and PR maintain that status using RECIST v1.1 as assessed by the investigator
The duration of progression free survival (PFS) defined as the length of time from first response (CR or PR) until the time of progressive disease (PD) identified using RECIST v1.1 as assessed by the investigator
Overall survival (OS) defined as the time from first dose of disitamab vedotin until time of death

Another secondary objective is to evaluate the pharmacokinetics (PK) of disitamab vedotin. The endpoints for this objective are select PK parameters of disitamab vedotin, total antibody (TAb) and unconjugated MMAE.

Another secondary objective is to evaluate the immunogenicity of disitamab vedotin. The endpoint for this objective is incidence of antidrug antibodies (ADA) against disitamab vedotin.

The exploratory objective is to assess biomarkers in relation to response, toxicity, pharmacodynamic relationship, or resistance to disitamab vedotin. Endpoints for this objective are biomarkers of disitamab vedotin-mediated biological activity, resistance, and their potential associations with clinical outcome measures.

### Inclusion/Exclusion Criteria

Subjects are eligible to be included in the study only if all the following criteria are met:
Age 18 years and older or considered an adult in the jurisdiction in which the study is taking place at the time of consent.
Cohort 1: HNSCC
   Must have pathologically-documented squamous cell carcinoma of the head and neck with primary tumor site arising from the oral cavity, oropharynx, hypopharynx, and larynx; tumors arising from the nasopharynx are excluded.
   Must have unresectable locally recurrent or metastatic stage disease.
   Prior therapies:
      Subjects must have disease progression after treatment with a platinum-based therapy:
         For recurrent disease not amenable to curative treatment or metastatic disease; or
         Experienced recurrence/progression within 6 months of last dose of platinum therapy administered as part of multimodal therapy in the curative setting
      No more than 1 line of cytotoxic chemotherapy for advanced disease
      May have received prior anti-PD(L)1 therapy
Cohort 2: NSCLC
   Must have pathologically documented NSCLC; subjects with small cell carcinoma elements are ineligible.
   Must have unresectable locally-advanced or metastatic stage disease.
   Prior therapies:
      Subjects must have progressed during or after a platinum-based therapy or, within 6 months of platinum-based adjuvant, neoadjuvant, or concomitant chemoradiotherapy for early or locally-advanced stage disease.
      Must have received prior anti-PD(L)1 therapy, unless contraindicated.
      Subjects with known EGFR, ALK, ROS or other actionable mutations must have received targeted therapy.
      Subjects with known HER2 mutations are ineligible.
      No more than 2 prior lines of cytotoxic chemotherapy for advanced disease.
   Cohort 3: ovarian cancer
      Must have pathologically documented epithelial cancers of ovarian, fallopian tube, or peritoneal origin
      Must have unresectable locally-advanced or metastatic stage disease.
      Prior therapies
         Subject must have platinum resistant disease (6 months or less between the completion of platinum-based treatment and identification of recurrence)
         Must not have received more than 4 lines of prior cytotoxic chemotherapies for advanced disease
         Subjects with known BRCA mutations are permitted, but subjects must have received targeted therapy with a PARP inhibitor
         May have received prior anti-PD(L)1 therapy
   Cohort 4: endometrial cancer
      Must have pathologically documented adenocarcinoma of the endometrium.
      Must have unresectable locally-advanced or metastatic stage disease.
      Prior therapies:
         Subjects must have relapsed/progressed after at least one prior platinum-based chemotherapy for recurrent, metastatic or primary unresectable disease. Subjects who experience disease progression less than 6 months after completion of adjuvant or neoadjuvant platinum-based chemotherapy are eligible
         Must not have received more than 3 lines of prior cytotoxic chemotherapies for advanced disease
         May have received prior anti-PD(L)1 therapy
   Disease demonstrating HER2 expression 1+, 2+, or 3+, as determined by local IHC testing on a fresh or archival tumor tissue. Use of the gastric cancer scoring algorithm is recommended. In each cohort of the signal-seeking Cohorts 1-4, if the first interim futility analysis is passed, additional 10 subjects with HER2 IHC 0 determined by local IHC testing are enrolled in this given cohort.
   Have measurable disease per RECIST v1.1 criteria as assessed by the investigator
   Subjects must be willing and able to provide formalin-fixed, paraffin-embedded (FFPE) tumor tissue blocks (or freshly sectioned slides, see laboratory manual for details), to a sponsor-designated central laboratory for biomarker analysis.
      Biology cohort only: baseline, on-treatment, and EOT biopsies are required.
         Subjects should have a tumor site that is accessible for biopsies and agree to biopsies per Section 8.5
         The biopsy lesion should be a separate lesion from tumor response assessment lesions.
   An ECOG Performance Status score of 0 or 1.
   Have adequate hepatic function as defined by the following:
      AST and ALT≤3× upper limit of normal (ULN) (≤5×ULN if liver metastases are present)
      Total bilirubin≤1.5×ULN. Exception: subjects with known history of Gilbert's syndrome with direct bilirubin≤1.5×ULN and normal AST and ALT are eligible
   Have adequate baseline hematologic parameters as defined by:
      Absolute neutrophil count (ANC)≥1.5×10⁹/L
      Platelet count ≥100×10⁹/L
      Hemoglobin ≥9.0 g/dL (≥90 g/L)
      In subjects transfused with any blood product before study entry, transfusion must be ≥14 days prior to start of therapy to establish adequate hematologic parameters independent from transfusion support
   Estimated glomerular filtration rate (GFR)≥30 mL/min/1.73 m² (measured by the Cockcroft-Gault formula, Modification of Diet in Renal Disease [MDRD] or 24-hour urine)
   Left ventricular ejection fraction (LVEF)≥50% as assessed by echocardiogram or MUGA documented within ≤3 months prior to first dose of study treatment
   Fridericia's corrected QT interval (QTcF)<470 ms
   Subjects of childbearing potential under the following conditions:
      Must have a negative serum, plasma, or urine pregnancy test (minimum sensitivity 25 mIU/mL or equivalent units of beta human chorionic gonadotropin [β-hCG]) result within 72 hours prior to the first dose of study intervention. Subjects with false positive results and documented verification that the subject is not pregnant are eligible for participation.
      Must agree not to try to become pregnant during the study and for at least 2 months after the final dose of disitamab vedotin.
      Must agree not to breastfeed or donate ova, from the time of informed consent and continuing through at least 2 months after the final dose of disitamab vedotin.
      If sexually active in a way that could lead to pregnancy, must consistently use at least 2 acceptable methods of birth control (contraception), at least 1 of which must be highly effective from the time of informed consent and continuing through at least 2 months after the final dose of disitamab vedotin.
   Subjects who can get someone pregnant (as defined in Section 10.4) under the following conditions:
      Must agree not to donate sperm from the time of informed consent and continuing through at least 4 months after the final dose of disitamab vedotin.
      If sexually active with a person of childbearing potential in a way that could lead to pregnancy, must consistently use at least 2 acceptable methods of birth control (contraception), at least 1 of which must be highly effective from the time of informed consent and continuing through at least 4 months after the final dose of disitamab vedotin.
      If sexually active with a person who is pregnant or breastfeeding, must consistently use a condom from the time of informed consent and continuing through at least 4 months after the final dose of disitamab vedotin.
   The subject or the subject's legally authorized representative must provide documented informed consent.

### Exclusion Criteria Include:

Prior treatment with an MMAE-containing agent.
Previous treatment with HER2-directed ADCs. Subjects previously treated with anti-HER2 monoclonal antibodies and/or HER2 tyrosine kinase inhibitors may be eligible for inclusion following consultation with the Sponsor.
Known hypersensitivity to any excipient contained in the drug formulation of disitamab vedotin.
History of another invasive malignancy within 2 years before the first dose of study intervention, or any evidence of residual disease from a previously diagnosed malignancy.
   Subjects with adequately resected early-stage non-melanoma skin cancer or carcinoma in situ are allowed.
   Subjects with a history of prostate cancer (T2NXMX or lower with Gleason score ≤7) treated with definitive intent (surgically or with radiation therapy) prior to study entry is acceptable, provided that the subject is considered prostate cancer free and the following criteria are met:
      Subjects who have undergone radical prostatectomy must have undetectable prostate specific antigen (PSA) at screening.
      Subjects who have had radiation must have a PSA doubling time >1 year (based on at least 3 values determined >1 month apart) and a total PSA value that does not meet Phoenix criteria for biochemical recurrence (i.e., <2.0 ng/ml above nadir).
Active CNS or leptomeningeal metastasis. Subjects with treated brain metastasis (surgery and/or radiotherapy) are eligible if they meet the following criteria:
   All known CNS lesions have been treated
   No evidence of clinical or radiographic disease progression in the CNS for ≥4 weeks after definitive treatment
   Neurological symptoms attributed to brain metastases have returned to baseline
   No steroids to manage symptoms related to CNS disease or its treatment within 28 days. Anti-convulsant treatment is allowed if the dose has been stable for 2 weeks.
Any active viral, bacterial, or fungal infection requiring active treatment within 7 days prior to the first dose of study intervention. Routine antimicrobial prophylaxis is permitted.
History of hepatitis B virus (HBV) infection (defined as positive for HBV surface antigen) or known active hepatitis C virus (HCV) infection (defined as HCV RNA [qualitative] is detected). Subjects who have been curatively treated for HCV infection are permitted if they have documented sustained virologic response. of ≥12 weeks. HBV negative DNA of ≥12 weeks is also allowed. No HBV or HCV testing is required, unless mandated by local regulations or institutional standard.
Known to be positive for human immunodeficiency virus (HIV). Testing is not required unless mandated by local regulations.
Documented history of a cerebral vascular event (stroke or transient ischemic attack), unstable angina, myocardial infarction, or cardiac symptoms consistent with New York Heart Association Class III or −IV congestive heart failure (CHF) within 6 months prior to the first dose of study intervention.
Major surgery within 2 weeks prior to the first dose of study intervention. Subject must have recovered adequately prior to Cycle 1 Day 1 (C1D1).
Any ongoing clinically significant toxicity (Grade ≥2, per the NCI CTCAE v5) associated with prior treatment (including systemic therapy, radiotherapy, or surgery) with exceptions of alopecia and anemia (Hemoglobin ≥9.0 g/dL is considered eligible).
Peripheral neuropathy ≥Grade 2.
Severe dyspnea at rest (NCI CTCAE Grade 3 or above) due to complications of advanced malignancy or baseline medical condition
Current therapy with other investigational agents.
Chemotherapy, radiotherapy, biologics, immunotherapy and/or other antitumor treatments within 2 weeks prior to first dose of study intervention.
Received a live or live-attenuated vaccine within 30 days prior of planned first dose of study intervention. Inactivated vaccines and other vaccine platforms are allowed.
Other serious underlying medical condition that, in the opinion of the investigator, would impair the subject's ability to receive or tolerate the planned treatment and follow-up.

### Example 2: Disitamab Vedotin-Based Extinction Coefficient (EC) Method

### Exemplary Protocol 1: Determination of Disitamab Vedotin Concentration by Slope Variable Pathlength Extension (SoloVPE) UV/Visible Spectroscopy

This example describes a method used to determine the concentration of disitamab vedotin using slope variable pathlength extension (SoloVPE) UV/visible spectroscopy. The SoloVPE system measures concentration based on the Beer-Lambert Law and the slope derived from absorbance measurements made at multiple path lengths.

### Equipment and Materials:

Agilent Cary 60 8453 UV/Vis spectrophotometer
C Technologies, Inc. slope variable pathlength extension (SoloVPE)
Quick Set Coupler Assembly (C Technologies p/n SA-SOLO-FTS)
SoloVPE fibrettes (C Technologies p/n OF0002)
Disposable small plastic vessel (C Technologies p/n OC0009-1)
Product-specific reference standard

### Procedure:

Instrument setup
If instrument is turned off, turn on and allow to warm up for 15-20 minutes. It is recommended to restart the instrument and computer at least once a week for consistent performance.
   Open the SoloVPE software and connect to the instrument.
   Perform a daily quick check test. Follow instructional prompts to determine system status, which should be “Excellent”.
   If system status is not “Excellent”, clean the Delivery Fiber and Detector Window with a folded Kimwipe by wiping a few times in one direction, followed by compressed air. Perform quick check again.
   Insert the fibrette until it stops, then allow the Quick Set Coupler Assembly to adjust the fibrette to the appropriate position. Insert a new fibrette for each sample.
Sample analysis
   Mix sample well before analysis.
   Add 100 μL of undiluted sample to a disposable vessel. If sample volume is limited, ≥30 μL is sufficient for analysis and may be recovered for future use. Note: Perform measurement immediately after adding sample to vessel.
   Place vessel with sample onto instrument vessel platform and slide the cover shield down.
   Select the ‘Quick Slope’ function from menu and enter settings:

| Parameter | Setting | |
|---|---|---|
| Sample vessel | PV-OC0009-1 (Disposable) | |
| Wavelength | 280 | nm |
| Extinction coefficient | 1.41 | mL mg⁻¹ cm⁻¹ |
| Baseline correction | Off | |
| Scatter correction | Single WL | |
| WL 1 (nm) | 320 | nm |

For replicate measurements, refer to table below. Use a new fibrette and vessel for each replicate.

| Testing Purpose | Number of replicates |
|---|---|
| Release and stability | 3 |
| Research and in-process | 2 |
| Assay control, if applicable | 3 |

### Calculations and Data Analysis:

The concentration will be calculated by the SoloVPE software, and results can be included in a report generated by the software. Alternatively, calculate the protein concentration using the following equation:

$$Concentration = \frac{{m}_{280}}{ε} \times \frac{10 mm}{1 cm}$$

Where: m₂₈₀=slope at 280 nm (mm⁻¹)
   ε=extinction coefficient
Calculate the average concentration from the duplicate or triplicate measurements to three decimal places.
For lyophilized samples: Calculate drug product content for lyophilized product vials using the following equation:

Drug product content=Concentration (mg/mL)×Final Reconstitution Volume (mL/vial)
Suitability Assessment
   Determine system and validity using the calculations and system criteria outlined in the table below. If the relevant suitability criteria is not met then results are not valid.

| | | Suitability |
|---|---|---|
| Testing Purpose | Calculation | Criteria |
| All | None | Each individual |
| | | replicate must |
| | | have an R² value |
| | | ≥ 0.999* |
| Release and stability | $\% RSD = ( \frac{{Stdev}_{( {C}_{a} , {C}_{b} , {C}_{c} )}}{{\overline{X}}_{( {C}_{a} , {C}_{b} , {C}_{c} )}} ) \times 100$ | % RSD ≤ 2% |
| | % RSD = percent relative standard deviation | |
| | Stdev = standard deviation | |
| | Cₐ = concentration for replicate 1 | |
| | C_{b} = concentration for replicate 2 | |
| | C_{c} = concentration for replicate 3 | |
| Research and in-process | $\% Difference = abs ( \frac{( {C}_{a} - {C}_{b} )}{{\overline{X}}_{( {C}_{a} , {C}_{b} )}} ) \times 100$ | % Difference ≤ 2% |
| | Cₐ = concentration for replicate 1 | |
| | C_{b} = concentration for replicate 2 | |
| Assay control | $\% Change = abs ( \frac{{C}_{1} - {C}_{2}}{{C}_{2}} ) \times 100$ | % Change ≤ 2.5% |
| | C₁ = average concentration of assay control | |
| | C₂ = CoT label concentration for assay | |
| | control | |

*If R² is < 0.999 and multiple points are not included in the regression, try adding a greater volume to a new vessel. Use a new fibrette.

Reporting results: Report the final concentration to the nearest 0.1 mg/mL and drug product content to the nearest 0.1 mg/vial.

### Exemplary Protocol 2: Determination of Disitamab Vedotin Concentration by Slope Variable Pathlength Extension (SoloVPE) UV/Visible Spectroscopy

This procedure describes the method used to determine the protein concentration of Disitamab monoclonal antibody (mAb) intermediate and Disitamab vedotin (ADC) by variable pathlength UV-Visible spectroscopy (SoloVPE).

This procedure is applicable to SoloVPE analysis of Disitamab mAb intermediate and Disitamab Vedotin DS and DP for in-process, lot release, and stability testing.

| Category | Objective |
|---|---|
| Applicability | SoloVPE |
| Scope | In-process, lot release, stability |
| Site | Canyon Park, North Creek |
| Product | Disitamab, Disitamab Vedotin |
| Material | mAb, DS, DP |
| Verification Level | Qualified |

Principle: The strength of Disitamab and Disitamab Vedotin are determined by SoloVPE. Sample is transferred to a vessel and the absorbance at 280 nm with scatter-correction at 320 nm is read over multiple pathlengths. This produces a slope (Abs/mm), which is then used to calculate protein concentration using Beer's law and a product specific extinction coefficient. Results are reported as mg of protein per mL of solution (mg/mL) for mAb and DS and as mg of protein per vial (mg/vial) for DP.

Equipment and Materials. NOTE: All equipment and materials are “or equivalent” unless otherwise noted.
Equipment:
   Calibrated pipettes and suitable tips
   Centrifuge
   Fibrettes (C Technologies, Inc. (CTI) Cat. No. OF0002-P50)
   Microcentrifuge Tubes, Eppendorf Polypropylene 1.5 mL (VWR Cat. No. 20901-551)
   SoloVPE System (CTI) with validated software, no substitution
      Agilent Cary 60 UV-Vis Spectrophotometer
      Quick Set Fibrette Coupler
   Vessel holders (CTI Cat. No. HM0178)
   Vessels, plastic (CTI Cat. No. OC0009-1) or fused silica (CTI Cat. No. OC0005-1)
Materials
   2% Hellmanex Cleaning Solution (LSR-0100 for fused silica vessels)
   Current Disitamab Reference Material, (no substitutions)
   Current Disitamab Vedotin Reference Material, (no substitutions)
   Water (Deionized (DI), Milli-Q, or HPLC Grade) (for fused silica vessels)
   Methanol, ACS grade or higher (for fused silica vessels)

### Sample Preparation

Disitamab
   In-process samples may be stored at 2-8° C. for up to a week after sampling. For all other samples, refer to their specification.
   Prior to use, equilibrate reference and samples to room temperature.
   Centrifuge an adequate volume to perform testing at 10,000 rcf for 5 minutes.
   Avoid disturbing any material at the bottom and transfer the supernatant into a new vial then mix by gentle inversion.
Disitamab Vedotin
   Prior to use, equilibrate reference and sample(s) to room temperature.
   Mix by gentle inversion.

### Sample Testing

Set up the instrument per OP-0385.
Open the Quick Slope window and select the settings defined below in Table 5.

TABLE 5
Quick Slope Settings

| | | | Ext. | | |
|---|---|---|---|---|---|
| | | | Coef. | | |
| | Sample | Wavelength | (mL/ | Baseline | Scatter |
| Product | Vessel | (nm) | mg*cm⁻¹) | Correction | Correction |
| Disitamab | Fused | 280 | 1.42 | Off | Single WL |
| Intermediate | Silica | | | | (320 nm) |
| (mAb) | or | | | | |
| | Plastic | | | | |
| Disitamab | Fused | 280 | 1.41 | Off | Single WL |
| Vedotin | Silica | | | | (320 nm) |
| (ADC) | or | | | | |
| | Plastic | | | | |

Read each vessel according to the Quick Slope settings above in Table 5.
   Use a new fibrette for each vessel. Replace the fibrette if it is removed from the vessel any time after placement into the solution.
   If using a fused silica vessel, use the same vessel for all readings. Clean the vessel between readings.
Prepare and analyze reference by transferring 100 μL into 3 separate vessel.
   Prepare vessels just prior to reading. Use reverse-pipetting to avoid bubble creation.
   If the reference fails to meet all validity requirements, prepare three new vessels and repeat analysis.
   If the reference fails to meet all validity requirements again, the run is invalid.
Prepare and analyze three vessels per sample by transferring 100 μL into separate vessels.
   Prepare vessels just prior to reading. Use reverse-pipetting to avoid bubble creation.
Save data.

### Calculations and Data Analysis

Use all significant FIGURES before rounding the final reported results.
Calculate the following:

| Material | Calculation |
|---|---|
| Reference Material | Individual protein concentration |
| | of triplicate preparations |
| | Average protein concentration of |
| | triplicate preparations |
| | % RSD of protein concentration |
| | % Difference from CoT |
| Samples mAb and DS | Individual protein concentration |
| (liquid) | of triplicate preparations |
| | Average protein concentration |
| | triplicate preparations |
| | % RSD of protein concentration |
| Sample DP | Individual protein concentration |
| (lyophilized) | of triplicate preparations |
| | Conjugate content of each |
| | individual preparation |
| | Average protein concentration |
| | triplicate preparations |
| | % RSD of conjugate content |

Calculations may be performed manually or by software (e.g. SoloVPE, Excel).
Use the following equations:
   Protein concentration:

$$Concentration ( mg / mL ) = \frac{{m}_{280}}{E C} \times \frac{10 mm}{1 cm}$$

Where:
ms280=Slope of sample at 280 nm
EC=Extinction coefficient (mL/mg*cm⁻¹) listed in Table 5
% Difference from CoT:

$$\vert \frac{Avg . protein conc . ( x . x ) - CoT conc . ( x . x )}{CoT conc . ( x . x )} \vert \times 100$$

Conjugate content for each replicate (mg/vial): Conc. (mg/mL)×Final Vol. (mL)=Conjugate Content (mg/vial) Where: Final vol. is the volume contribution from dissolved solids (per the product specification) plus the actual reconstitution volume used.

### System Suitability Acceptance Criteria

The R2 value for each measurement must be ≥0.999.
All ten points must be displayed as ‘Y’ in the ‘In Reg’ column for linear regression.
The % RSD for concentration of the triplicate preparations must be ≤1.5%.
The average protein concentration must be within 5.0% of the concentration stated on the
Certificate of Testing (CoT).
If any of the above criteria are not met, the assay is invalid.

### Sample Acceptance Criteria

The R2 value for each measurement must be ≥0.999.
All ten points must be displayed as ‘Y’ in the ‘In Reg’ column for linear regression.
The % RSD for concentration of the triplicate preparations must be:
   mAb intermediate, DS, and DP samples: ≤1.5%.
   In-process samples must meet the appropriate criteria in the following table.

| Sample (mg/mL) | % RSD Criteria |
|---|---|
| X > 1.0 | ≤1.5% |
| X ≤ 1.0 | ≤3.0% |

If any of the above criteria are not met, the sample is invalid.

### Reportable Results

The reportable result is the average of triplicate preparations.
Liquid mAb and DS:
   Report average protein concentration (in mg/mL) per the appropriate specification document.
Lyophilized DP:
   Report the average conjugate content (in mg/vial) per the appropriate specification document.

### Example 3: A Phase 2 Basket Study of Disitamab Vedotin in Adult Subjects with Previously Treated, Locally-Advanced Unresectable or Metastatic Solid Tumors That Express HER2

### Design

This example describes a single-group, multi-cohort, multicenter, interventional, open-label Phase 2 study designed to assess the activity, safety, and tolerability of disitamab vedotin monotherapy for the treatment of subjects with previously treated, LA/m solid tumors that express HER2.

The single experimental arm will receive disitamab vedotin monotherapy (also known as RC48, RC48-ADC), given into the vein (IV, intravenous) every 2 weeks. Subjects are enrolled into separate cohorts based on tumor type. There are 4 tumor specific cohorts: HNSCC (Cohort 1), NSCLC (Cohort 2), ovarian cancer (Cohort 3), and endometrial cancer (Cohort 4). There will be approximately 160 anticipated subjects enrolled.

### Objectives and Endpoints

In this study, the primary outcome measure is confirmed objective response rate per the Response Evaluation in Solid Tumors version 1.1 (RECIST v1.1) as assessed by the investigator, i.e., the proportion of patients who achieve a confirmed complete response (CR) or partial response (PR) according to RECIST v1.1 as assessed by the investigator (time frame: 3 years).

### Secondary Outcome Measures Include:

Type, incidence, severity, seriousness, and relatedness of AEs including AEs of special interest (AESIs), i.e., number of participants with adverse events (AE): any untoward medical occurrence in a clinical study participant, temporally associated with the use of study intervention, whether or not considered related to the study intervention (time frame: through 30-37 days after the last dose of DV; approximately 5 years).
Type, incidence, and severity of laboratory abnormalities as well as significant changes from baseline, i.e., number of participants with laboratory abnormalities (time frame: through 30-37 days after the last dose of DV; approximately 5 years).
Frequency of treatment interruptions, dose reductions and treatment discontinuations due to AEs, i.e., number of participants with dose alterations due to AEs (time frame: 5 years).
Confirmed disease control rate (DCR) per RECIST v1.1 as assessed by the investigator: the proportion of participants with stable disease (SD) or confirmed CR or PR according to RECIST v1.1 (time frame: approximately 5 years).
Duration of response (DOR) per RECIST v1.1 as assessed by the investigator, i.e., the time from state of the first documentation of objective tumor response of CR or PR (that is subsequently confirmed) to the first documentation of progressive disease (PD) per RECIST v1.1, or to death due to any cause (time frame: 5 years).
Progression free survival (PFS) per RECIST v1.1 as assessed by the investigator, i.e., the time from the start of the study treatment or the first documentation of PD per RECIST v1.1 or death due to any cause, whichever occurs first (time frame: 5 years).
Overall survival (OS): the time from the start of study treatment to the date of death due to any cause (time frame: approximately 5 years).
Select PK parameters of disitamab vedotin, total antibody (TAb) and unconjugated MMAE, wherein the pharmacokinetic (PK) parameter is:
   the area under the concentration-time curve to the time of the last quantifiable concentration (AUClast) analyzed through cycle 2 (time frame: approximately 1 month),
   the maximum concentration (Cmax) analyzed through end of treatment (time frame: through 30-37 days after the last dose of DV; approximately 5 years), and/or
   the trough concentration (Ctrough) analyzed through end of treatment (time frame: through 30-37 days after the last dose of DV; approximately 5 years).
Incidence of antidrug antibodies (ADA) against disitamab vedotin (time frame: through 30-37 days after the last dose of DV; approximately 5 years).

### Inclusion/Exclusion Criteria

### Key Eligibility Criteria Include:

Subjects of all sexes are eligible.
At least 18 years of age
Cohort 1: Head and neck squamous cell carcinoma (HNSCC)
   Pathologically-documented squamous cell carcinoma of the head and neck with primary tumor site arising from the oral cavity, oropharynx, hypopharynx, and larynx
   Unresectable locally recurrent or metastatic stage disease
   Prior therapies:
      Participants must have disease progression after treatment with a platinum-based therapy.
      No more than 1 line of cytotoxic chemotherapy for advanced disease
Cohort 2: Non-small cell lung cancer (NSCLC)
   Pathologically documented NSCLC
   Unresectable locally-advanced or metastatic stage disease
   Prior therapies
      Must have progressed during or after a platinum-based therapy or, within 6 months of platinum-based adjuvant, neoadjuvant, or concomitant chemoradiotherapy for early or locally-advanced stage disease
      Must have received prior anti-PD(L)1 therapy, unless contraindicated
      No more than 2 prior lines of cytotoxic chemotherapy for advanced disease
Cohort 3: Ovarian Cancer
   Pathologically documented epithelial cancers of ovarian, fallopian tube, or peritoneal origin
   Unresectable locally-advanced or metastatic stage disease
   Prior therapies
      Must have platinum resistant disease (6 months or less between the completion of platinum-based treatment and identification of recurrence)
      Must not have received more than 4 lines of prior cytotoxic chemotherapies for advanced disease
      May have received prior anti-PD(L)1 therapy
Cohort 4: Endometrial Cancer
   Must have pathologically documented adenocarcinoma of the endometrium
   Must have unresectable locally-advanced or metastatic stage disease.
   Prior therapies
      Must have relapsed/progressed after at least one prior platinum-based chemotherapy for recurrent, metastatic or primary unresectable disease.
      Must not have received more than 3 lines of prior cytotoxic chemotherapies for advanced disease
      May have received prior anti-PD(L)1 therapy
HER2 expression 1+, 2+, or 3+, as determined by local IHC testing on a fresh or archival tumor tissue. Note: Subjects with HER2 mutations are eligible.
Have measurable disease per RECIST v1.1 criteria as assessed by the investigator
Able to provide formalin-fixed, paraffin-embedded (FFPE) tumor tissue blocks (or freshly sectioned slides).
Eastern Cooperative Oncology Group (ECOG) Performance Status score of 0 or 1.

### Exclusion Criteria Include:

Prior treatment with an MMAE-containing agent.
Known hypersensitivity to any excipient contained in the drug formulation of disitamab vedotin.
History of another invasive malignancy within 2 years before the first dose of study intervention, or any evidence of residual disease from a previously diagnosed malignancy.
Active CNS or leptomeningeal metastasis.

### Example 4: A Phase 2 Basket Study of Disitamab Vedotin in Adult Subjects with Previously Treated, Locally-Advanced Unresectable or Metastatic Solid Tumors That Express HER2

Subjects are enrolled into separate cohorts based on tumor type. There are 4 tumor specific cohorts: HNSCC (Cohort 1), NSCLC (Cohort 2), ovarian cancer (Cohort 3), and endometrial cancer (Cohort 4). Approximately 160 subjects will be enrolled in the study.

The investigational medicine product is disitamab. Disitamab vedotin is administered as an IV infusion every 2 weeks. Disitamab vedotin is formulated as a lyophilized powder for infusion.

The study is non-randomized and open, i.e., unblinded.

### Objectives and Endpoints

The primary objective of this study is to evaluate the antitumor activity of disitamab vedotin in subjects with previously treated, locally-advanced unresectable or metastatic (LA/m) HER2 expressing solid tumors. The primary efficacy assessment is confirmed objective response rate (ORR) per Response Evaluation Criteria in Solid Tumors version 1.1 (RECIST v1.1) as assessed by the investigator.

### Secondary Objectives of the Study Include:

To evaluate the safety and tolerability profile of disitamab vedotin
To assess antitumor activity of disitamab vedotin per investigator assessment by other clinically relevant measures
To evaluate the pharmacokinetics (PK) of disitamab vedotin
To evaluate the immunogenicity of disitamab vedotin

### Secondary Efficacy Assessments Include:

Type, incidence, severity, seriousness, and relatedness of adverse events (AEs) including AEs of special interest (AESIs)
Type, incidence, and severity of laboratory abnormalities as well as significant changes from baseline
Frequency of treatment interruptions, dose reductions and treatment discontinuations due to AEs
Confirmed disease control rate (DCR) per RECIST v1.1 as assessed by the investigator.
Duration of response (DOR) per RECIST v1.1 as assessed by the investigator
Progression free survival (PFS) per RECIST v1.1 as assessed by the investigator
Overall survival (OS)
Select PK parameters of disitamab vedotin, total antibody (TAb) and unconjugated MMAE
Incidence of antidrug antibodies (ADA) against disitamab vedotin

### Inclusion/Exclusion Criteria

### Inclusion Criteria Include:

### 1. Cohort 1: HNSCC

Pathologically-documented squamous cell carcinoma of the head and neck with primary tumor site arising from the oral cavity, oropharynx, hypopharynx, and larynx
Unresectable locally recurrent or metastatic stage disease
Prior therapies:
   Participants must have disease progression after treatment with a platinum-based therapy
   No more than 1 line of cytotoxic chemotherapy for advanced disease

### Cohort 2: NSCLC

Pathologically documented NSCLC
Unresectable locally-advanced or metastatic stage disease
Prior therapies
   Must have progressed during or after a platinum-based therapy or, within 6 months of platinum-based adjuvant, neoadjuvant, or concomitant chemoradiotherapy for early or locally-advanced stage disease
   Must have received prior anti-PD(L)1 therapy, unless contraindicated
   No more than 2 prior lines of cytotoxic chemotherapy for advanced disease

### Cohort 3: Ovarian Cancer

Pathologically documented epithelial cancers of ovarian, fallopian tube, or peritoneal origin
Unresectable locally-advanced or metastatic stage disease
Prior therapies
   Must have platinum resistant disease (6 months or less between the completion of platinum-based treatment and identification of recurrence)
   Must not have received more than 4 lines of prior cytotoxic chemotherapies for advanced disease
   May have received prior anti-PD(L)1 therapy

### Cohort 4: Endometrial Cancer

Must have pathologically documented adenocarcinoma of the endometrium
Must have unresectable locally-advanced or metastatic stage disease.
Prior therapies
   Must have relapsed/progressed after at least one prior platinum-based chemotherapy for recurrent, metastatic or primary unresectable disease
   Must not have received more than 3 lines of prior cytotoxic chemotherapies for advanced disease
   May have received prior anti-PD(L)1 therapy
2. HER2 expression of 1+, 2+, or 3+, as determined by local IHC testing on a fresh or archival tumor tissue. Note: Subjects with HER2 mutations are eligible.
3. Have measurable disease per RECIST v1.1 criteria as assessed by the investigator
4. Able to provide formalin-fixed, paraffin-embedded (FFPE) tumor tissue blocks (or freshly sectioned slides)
5. Eastern Cooperative Oncology Group (ECOG) performance status score of 0 or 1

### Exclusion Criteria Include:

1. Prior treatment with an MMAE-containing agent.
2. Known hypersensitivity to any excipient contained in the drug formulation of disitamab vedotin.
3. History of another invasive malignancy within 2 years before the first dose of study intervention, or any evidence of residual disease from a previously diagnosed malignancy.
4. Active untreated CNS or leptomeningeal metastasis

### Example 5: Bovine Serum Albumin (BSA)-Based Extinction Coefficient (EC) Method

This example describes a method used to determine the concentration of disitamab vedotin using a bovine serum albumin-based (BSA-based) UV spectrophotometric method that determines the protein concentration using the absorbance of the sample and an extinction coefficient (EC).

The Lowry colorimetric method was previously used to determine disitamab vedotin concentration using BSA as the reference standard. Alternatively, UV spectrophotometric methods were also previously developed to determine the protein concentration using the absorbance of the sample and an extinction coefficient. The BSA-based extinction coefficient method used in the present application uses an EC value that was established mathematically to align the Lowry colorimetric and UV spectrophotometric methods, such that both yield the same estimated measurements of disitamab vedotin protein concentration.

Briefly, the sample is transferred to a vessel, and the absorbance at 280 nm with scatter-correction at 320 nm is read over multiple pathlengths using a SoloVPE system. This produces a slope (Abs/mm) which is then used to calculate protein concentration using Beer's law and a product-specific extinction coefficient.

### Calculations and Data Analysis:

The concentration will be calculated by the SoloVPE software, and results can be included in a report generated by the software. Alternatively, calculate the protein concentration using the following equation:

$$Concentration = \frac{{m}_{280}}{ε} \times \frac{10 mm}{1 cm}$$

Where: m₂₈₀=slope at 280 nm (mm⁻¹)
   ε=extinction coefficient
Calculate the average concentration from the duplicate or triplicate measurements to three decimal places.
For lyophilized samples: Calculate drug product content for lyophilized product vials using the following equation:

$$Drug product content = Concentration ( mg / mL ) \times Final Reconstitution Volume ( mL / vial )$$

1. A method for treating or preventing progression of cancer in an individual, comprising administering to the individual an effective amount of an anti-human epidermal growth factor receptor 2 (HER2) antibody-drug conjugate that comprises an anti-HER2 antibody and a cytotoxic molecule;
wherein the anti-HER2 antibody comprises a heavy chain comprising a heavy chain variable (VH) domain and a light chain comprising a light chain variable (VL) domain; wherein
the VH domain comprises a CDR-H1 comprising the amino acid sequence DYYIH (SEQ ID NO: 1), a CDR-H2 comprising the amino acid sequence RVNPDHGDSYYNQKFKD (SEQ ID NO: 2), and a CDR-H3 comprising the amino acid sequence NYLFDH (SEQ ID NO:3); and
wherein the VL domain comprises a CDR-L1 comprising the amino acid sequence KASQDVGTAVA (SEQ ID NO:4), a CDR-L2 comprising the amino acid sequence WASIRHT (SEQ ID NO:5), and a CDR-L3 comprising the amino acid sequence HQFATYT (SEQ ID NO: 6); and
wherein the cancer is selected from the group consisting of: head and neck squamous cell carcinoma (HNSCC), ovarian cancer, and endometrial cancer.

2. (canceled)

3. A method for treating or preventing progression of a cancer in an individual, comprising administering to the individual an effective amount of an anti-human epidermal growth factor receptor 2 (HER2) antibody-drug conjugate that comprises an anti-HER2 antibody and a cytotoxic molecule;
wherein the anti-HER2 antibody comprises a heavy chain comprising a heavy chain variable (VH) domain and a light chain comprising a light chain variable (VL) domain; wherein
the VH domain comprises a CDR-H1 comprising the amino acid sequence DYYIH (SEQ ID NO: 1), a CDR-H2 comprising the amino acid sequence RVNPDHGDSYYNQKFKD (SEQ ID NO: 2), and a CDR-H3 comprising the amino acid sequence NYLFDH (SEQ ID NO:3); and
wherein the VL domain comprises a CDR-L1 comprising the amino acid sequence KASQDVGTAVA (SEQ ID NO:4), a CDR-L2 comprising the amino acid sequence WASIRHT (SEQ ID NO:5), and a CDR-L3 comprising the amino acid sequence HQFATYT (SEQ ID NO: 6); and
wherein the cancer is non-small cell lung cancer (NSCLC), and
wherein, prior to administration of the antibody-drug conjugate, the individual (a) progressed during or after a platinum-based therapy or, within 6 months of platinum-based adjuvant, neoadjuvant, or concomitant chemoradiotherapy for early or locally-advanced stage disease; or (b) received prior anti-PD(L)1 therapy.

4. (canceled)

5. The method of claim 3, wherein, prior to administration of the antibody-drug conjugate, the individual (a) progressed during or after a platinum-based therapy or, within 6 months of platinum-based adjuvant, neoadjuvant, or concomitant chemoradiotherapy for early or locally-advanced stage disease; and (b) received prior anti-PD(L)1 therapy.

6. The method of claim 1, wherein the VH domain comprises the amino acid sequence of SEQ ID NO:7.

7. The method of claim 1, wherein the VL domain comprises the amino acid sequence of SEQ ID NO:8.

8. (canceled)

9. (canceled)

10. The method of claim 1, wherein the cytotoxic molecule comprises a tubulin inhibitor or DNA damaging agent.

11. The method of claim 10, wherein the tubulin inhibitor comprises a dolastatin or derivative thereof, auristatin or derivative thereof, or maytansinoid or derivative thereof.

12. The method of claim 11, wherein the tubulin inhibitor comprises monomethyl auristatin E (MMAE), monomethyl auristatin F (MMAF), or auristatin F (AF).

13. (canceled)

14. (canceled)

15. (canceled)

16. (canceled)

17. (canceled)

18. (canceled)

19. The method of claim 1, wherein the antibody-drug conjugate is disitamab vedotin.

20. (canceled)

21. The method claim 1, wherein the cancer has been previously treated with a HER2 directed antibody-drug conjugate.

22. (canceled)

23. The method of claim 1 wherein:
prior to administration of the antibody-drug conjugate, the individual
(a) progressed during or after ≥1 prior line of systemic therapy for LA/m disease and
(b) progressed during or after, or is intolerant of, the most recent line of systemic therapy.

24. The method of claim 1, wherein the cancer is HNSCC with a primary tumor site arising from the oral cavity, oropharynx, hypopharynx, or larynx.

25. The method of claim 3, wherein the cancer is NSCLC with a mutation in EGFR, ALK, or ROS, and the individual has received prior targeted therapy.

26. The method of claim 1, wherein the cancer is ovarian cancer comprising epithelial cancers of ovarian, fallopian tube, or peritoneal origin.

27. (canceled)

28. (canceled)

29. (canceled)

30. (canceled)

31. (canceled)

32. (canceled)

33. (canceled)

34. The method of claim 1, wherein the antibody-drug conjugate is administered to the individual at a dose of 1.5 mg/kg.

35. (canceled)

36. The method of claim 34, wherein the dose is measured using the disitamab vedotin-based Extinction Coefficient (EC) method.

37. (canceled)

38. The method of claim 1, wherein the antibody-drug conjugate is administered to the individual every 2 weeks or every 14 days.

39. The method of claim 1, wherein the antibody-drug conjugate is disitamab vedotin, and the method comprises intravenously administering disitamab vedotin to the individual at 1.5 mg/kg with a maximum dose of 150 mg every two weeks or every 14 days, optionally wherein the dose is measured using the disitamab vedotin-based Extinction Coefficient (EC) method.

40. The method of claim 1, wherein administration of the antibody-drug conjugate results in a complete response (CR) or partial response (PR) in the individual.

41. (canceled)

42. (canceled)

43. (canceled)

44. (canceled)

45. A pharmaceutical composition for use in treating a previously treated cancer in an individual, wherein the use comprises administering an effective amount of the anti-HER2 antibody-drug conjugate according to the method of claim 1.
